(19) United States
(12) Patent Application Publication
(10) Pub. No.: US 2021/0016283 A1
(43) Pub. Date: Jan. 21, 2021

(54) ULTRAHIGH THROUGHPUT PROTEIN DISCOVERY

(71) Applicant: Arbor Biotechnologies, Inc., Cambridge, MA (US)

(72) Inventors: Huaibin ZHANG, Wayland, MA (US); Shaorong CHONG, Arlington, MA (US); David A. SCOTT, San Francisco, CA (US); Winston X. YAN, Brookline, MA (US)

(21) Appl. No.: 16/980,147

(22) PCT Filed: Mar. 12, 2019
(86) PCT No.: PCT/US19/21765

§ 371 Date: Sep. 11, 2020

Related U.S. Application Data
(60) Provisional application No. 62/641,940, filed on Mar. 12, 2018.

(51) Int. Cl. B01L 3/00; B01D 63/08

(52) U.S. Cl. CPC B01L 3/502753; B01D 63/088; B01L 2300/0858; B01L 2200/0642; B01L 2300/0851; B01L 2200/0668

(57) ABSTRACT

The disclosure relates to methods and systems for ultrahigh throughput protein synthesis and analysis.

### CLAIM OF PRIORITY

This application claims the benefit of U.S. Provisional Application No. 62/641,940, filed on Mar. 12, 2018. The entire contents of the foregoing are incorporated herein by reference.

### TECHNICAL FIELD

The disclosure relates to methods and systems for high throughput protein discovery.

### BACKGROUND

As genome sequences of various organisms have become available, it is now possible to analyze protein functions on a genome-wide scale. Structural genomics and proteomics, therefore, have become major research foci. Several protein function screening platforms have been developed and used for various purposes, e.g., developing novel antibiotics and novel cancer therapies. However, these platforms have various limitations that prevent them from being used to characterize the large number of proteins, peptides, and enzymes that have been uncovered by genomic and metagenomic sequencing efforts. For example, in vivo cell-based platforms for protein expression and evaluation take advantage of the cell as a natural environment for efficient protein production and functional assays.

Such platforms based on prokaryotic organisms such as Escherichia coli or eukaryotic model cells such as Human Embryonic Kidney cells are often favored for being well characterized and simple to manipulate. However, using conventional methods of protein extraction and purification, the number of proteins that can be synthesized and studied is limited compared to the scale of proteins identified from genomic and metagenomic sequencing. Pooled screening techniques that enable the simultaneous testing of multiple constructs at once suffer from limitations of readouts that cannot adequately measure a diverse set of protein functions and separate functional from non-functional protein candidates within a pool. Furthermore, all of such cell-based methods are limited in that a significant number of proteins cannot be adequately expressed in vivo—for example, expressing heterologous proteins in E. coli often leads to insoluble aggregated folding intermediates, known as inclusion bodies.

There remains a need for an ultra-high throughput protein discovery platform to address pressing needs in human health, sustainability, and beyond.

### SUMMARY

The disclosure provides systems and methods of leveraging genomic and metagenomic sequencing with large-scale gene synthesis, non-cellular protein synthesis, and low volume protein functional assays for ultrahigh throughput protein discovery and characterization. The systems and methods described in the present disclosure can perform 100,000s or more reactions per run and importantly, screen a diverse and versatile set of protein activities across a number of different domains and applications, including but not limited to genome editing, biologic drug discovery, agricultural insecticides, and advancing environmental sustainability. Such identified proteins can provide novel biotechnological applications, as well as add additional diversity of features and versatility to known protein activities.

In one aspect, the disclosure features microwell array systems having a microwell array including a plurality of isolated microwells, each microwell having side walls, a bottom wall, and a top opening, wherein the microwells are positioned in an array, and wherein each well comprises one or more filter holes arranged in the bottom wall of the microwell; a cover, e.g., a movable plate or immiscible fluid, arranged to optionally and selectively cap one or more of the filter holes; a reservoir to receive waste liquids exiting the microwells through the filter holes, through the top opening of microwells, or both; a substrate to receive contents of one or more of the microwells deposited at one or more locations of a microarray (also known as a “blotting plate”), wherein each location has a known coordinate within the microarray; a system for adding liquids to each microwell; a system for adding microbeads to each microwell; and a system for selecting and marking selected contents at specified locations in the microarray, and optionally, a system to decode contents with given coordinates.

In some embodiments, the volume of each isolated microwell is about 0.5 picoliters to about 100 nanoliters (nl), each microwell has a diameter of from about 5 to 200 microns, and each filter hole has a diameter of from about 0.5 to 150.0 microns. In certain embodiments, the volume of each isolated microwell is less than 100 nanoliters (nl), 50 nl, 10 nl, 5 nl, 1 nl, 500 picoliters (pl), 250 pl, 100 pl, 50 pl, 25 pl, 20 pl, 15 pl, 10 pl, 5 pl, or 1 pl.

In some implementations, each microwell has a diameter of less than 200, 150, 100, 90, 80, 70, 60, 50, 40, 30, 20, or 10 microns and each filter hole has a diameter of about 0.5, 1, 2, 3, 4, 5, 6, 7, 8, 9, 10, 15, 20, 25, 50, 75, 100, 125, or 150 microns.

In some embodiments, the microwell array has at least 5K, 10K, 50K, 100 K, 250 K, 500 K, 1 M, 5 M, 10 M, or 15 M microwells. In some embodiments, the microwell array has at least 100, 1 K, 5 K, 10 K, 50K, or 100 K microwells per cm².

In certain embodiments, the inner walls of each microwell are hydrophilic, and surfaces of the microwell array and of the cover are hydrophobic.

In some implementations, the system for adding liquids adds liquids to each microwell via capillary force.

In certain implementations, the system for adding liquids includes one or more microfluidic channels. In some embodiments, the system for adding liquids includes a liquid jetting system. In some embodiments, the system for adding liquids includes a pressure or vacuum pump.

In some implementations, the system for adding liquids to each microwell includes a motor arranged to rotate the microwell array to distribute a liquid across a surface of the microwell array and into each microwell by spin-coating.

In certain implementations, the motor is controlled to spin sufficiently fast to remove excess liquids once the microwells are filled by the liquids.

In some embodiments, the diameter of the filter holes is smaller than a diameter of beads used with the system.

In certain embodiments, each microwell includes two or more filter holes, wherein all filter holes are smaller than a diameter of beads used with the system and wherein second and any subsequent filter holes are smaller than the first filter hole.

In some embodiments, the hole (e.g., rectangular or triangular shape) cannot be sealed completely by the beads and the remaining gap serve as a draining port for liquid.

In some implementations, the protein screening system further includes a centrifugation system arranged to empty waste liquids in the microwells by centrifugation.

In certain implementations, the liquid in each microwell is deposited on the substrate by centrifugation or air pressure.

In some embodiments, the liquids are reagents used for screening including emulsions, suspensions, and cell-free protein synthesis reagents.

In certain embodiments, each microwell includes one filter hole, wherein the filter hole is smaller than a diameter of beads. The hole (e.g., rectangular, triangular, or other shape) would not be sealed completely by the beads and the gap left serve as a draining port for liquid.

In another aspect, the disclosure provides methods of identifying a nucleic acid molecule encoding a polypeptide and/or RNA having a desired bioactivity. The methods include:

(a) attaching a plurality of nucleic acid constructs to a plurality of beads;

(b) loading the plurality of beads into microwells in a microwell array, e.g., the microwell array system described herein, wherein each microwell in the microwell array receives one or more beads, e.g., at most one bead;

(c) incubating the nucleic acid constructs with in vitro transcription/translation (IVTT) reagents for a time sufficient to produce a plurality of polypeptides encoded by the nucleic acid constructs in the microwell array;

(d) depositing nucleic acid constructs or polypeptides from each microwell in the microwell array at specific discrete locations on a substrate to form a ‘blotting plate’ of nucleic acid constructs or polypeptides preserving the spatial relationship of the samples, wherein each location in the blotting plate has a known coordinate that corresponds to a specific microwell in the microwell array;

(e) determining a bioactivity of the polypeptides and/or RNA in the microwells or on the blotting plate and selecting a microwell or location on the blotting plate corresponding to a desired bioactivity; and

(g) determining which nucleic acid constructs correspond to the selected microwell or location on the blotting plate corresponding to the desired bioactivity, thereby identifying the nucleic acid construct that corresponds to the polypeptide and/or RNA having the desired bioactivity.

In some embodiments, the methods further include assembling the plurality of nucleic acid constructs in each microwell by releasing oligo fragments of the nucleic acid constructs and assembling the oligo fragments, e.g., by polymerase cycling assembly, Golden gate assembly, or Gibson assembly.

In certain embodiments, each bead is bound to one or more nucleic acid constructs.

In some implementations, the one or more nucleic acid constructs at the location on the substrate that corresponds to the microwell containing the polypeptide having the desired bioactivity is selected by light induced DNA trapping, light induced surface charge switch, light induced pH change, light induced dissociation, laser microdissection, micromanipulator, or other mechanic picking method.

In certain implementations, the one or more nucleic acid constructs at the location on the substrate that corresponds to the microwell containing the polypeptide having the desired bioactivity is selected by sealing the nucleic acid construct by a sealing reagent.

In some embodiments, the one or more nucleic acid constructs at the location on the substrate that corresponds to the microwell containing the polypeptide having the desired bioactivity is selected by hybridizing the nucleic acid construct with a set of fluorescence probes.

In certain embodiments, the one or more nucleic acid constructs at the location of the substrate that corresponds to the microwell containing the polypeptide having the desired bioactivity is selected by a light-activated nuclease that releases the one or more nucleic acid constructs into solution for collection and sequencing to identify the constructs that correspond to the polypeptides that exhibit the desired bioactivity.

In some implementations, the one or more nucleic acid constructs at the location of the substrate that corresponds to the microwell containing the polypeptide having the desired bioactivity is selected automatically by the polypeptide catalyze a reaction that generates air bubble to expel liquid containing nucleic acid out from the microwells.

In certain implementations, the one or more nucleic acid constructs at the location of the substrate that corresponds to the microwell containing the polypeptide having the desired bioactivity is selected automatically by the polypeptide catalyze a reaction or condition that deforms or dissolves the beads so that nucleic acid could passing through the filtering holes.

In some embodiments, the bioactivity of the polypeptide is analyzed by a catalytical reaction, a binding assay, and a cleavage assay resulting optical signals (e.g., fluorescence, absorption).

In one aspect, the disclosure also provides methods of adding a liquid to a plurality of isolated microwells on a microwell array. The methods include applying a liquid to the microwell array; rotating the microwell array at a first speed, thereby filling each microwell on the microwell array with the liquid; and rotating the microwell array at a second speed, thereby removing excess liquid on the top of microwell array.

In some embodiments, the first speed is slower than the second speed. In certain embodiments, the liquid is applied to the microwell array continuously.

In some implementations, the liquid contains a plurality of beads. In certain implementations, the excess liquid that is removed from the microwell array is less than 1%, 2%, 3%, 4%, 5%, 6%, 7%, 8%, 9%, 10%, 20%, 30%, 40%, 50%, 60%, 70%, or 80% of total liquid that is applied to the microwell array.

In another aspect, the disclosure features a centrifuge system having: a support for a microwell array, wherein the support is arranged for rotation and comprises a plate configured for connection to a microwell array and for receiving liquids from the microwell; a liquid dispenser positioned above a surface of the microwell array and configured to dispense one or more liquids onto the center of the surface of the microarray; a first motor arranged to rotate the support around a central axis (812) of the microwell array connected to the support when the microwell array is in a horizontal position; and a second motor arranged to move the microwell array into a vertical position, and to rotate the microwell array around an axis (912) that perpendicular to the central axis (812) of the microwell array and parallel to the vertically positioned microwell array surface.

In some embodiments, microwell array comprises a plurality of isolated microwells, each microwell having side walls, a bottom wall, and a top opening, and wherein each microwell comprises one or more filter holes arranged in the bottom wall of the microwell.

In certain embodiments, the volume of each isolated microwell is about 0.5 picoliters to about 50 nanoliters (nl), e.g., 1, 5, 10, 25, 50, 75, 100, 250, 500, 750, 1000 picoliters or 1, 5, 10, 20, 30, 40, or 50 nanoliters, each microwell has a diameter of from about 5 to 200 microns, e.g., 5, 10, 15, 25, 50, 75, 100, 150, or 200 microns, and each filter hole has a diameter of from about 0.5 to 40.0 microns, e.g., 0.5, 1.0, 5.0, 10.0, 20.0, 30.0, or 40.0 microns.

In some implementations, the first motor is controlled to rotate the support sufficiently fast to remove excess liquids from the surface of the microwell array once the microwells are filled by the liquids.

In certain implementations, the second motor is controlled to spin sufficiently fast to move liquids out of microwells through the filter holes and onto the plate.

In one aspect, the disclosure also provides methods of selectively releasing one or more nucleic acid constructs from a substrate (e.g., plate, beads, microwell array). The methods include providing a substrate comprising an array of nucleic acid constructs; adding a photosensitive agent to the substrate; exposing one or more selected locations on the substrate to light, wherein the light induces the photosensitive agent to cross-link the polymer layer at the selected locations, thereby trapping nucleic acid constructs at the selected locations within the substrate; and washing the substrate with a wash solution, thereby releasing one or more nucleic acid constructs from unselected locations.

In some embodiments, the one or more selected locations are exposed to light by using a light projector with a predetermined pattern. In certain embodiments, the substrate plate is covered by a photomask, and the one or more selected locations are exposed to light by uncovering portions of the photomask at the selected locations.

In some implementations, the methods further include sequencing the one or more nucleic acid constructs in the wash solution. In certain implementations, the methods further include releasing and sequencing the nucleic acid constructs that are trapped by the cross-linked polymer.

In another aspect, the disclosure also provides methods of selectively releasing one or more nucleic acid constructs from a surface. The methods include providing a surface comprising an array of nucleic acid constructs, wherein the nucleic acid constructs are attached to the surface through an electronic charge interaction; and exposing one or more selected locations on the surface to light, wherein the light induces charge-switching of the surface, thereby releasing nucleic acid constructs at the selected locations on the surface.

In some embodiments, one or more selected locations are exposed to light by using a light projector with a predetermined pattern. In certain embodiments, the plate is covered by a photomask, and the one or more selected locations are exposed to light by uncovering portions of the photomask at the selected locations.

In some implementations, the methods further include sequencing the one or more nucleic acid constructs that are released from the plate. In certain implementations, the methods further include releasing and sequencing the nucleic acid constructs at unselected locations.

In one aspect, the disclosure further relates to methods for loading of beads into microwells such that microwells contain either one or no beads, and that a low percentage of the microwells contain two or more beads. The methods include obtaining a plurality of beads in a liquid; obtaining a microwell array system described herein, wherein each microwell comprises one or more larger filter holes and one or more smaller filter holes; wherein each larger filter hole has a diameter that is smaller than a smallest outer diameter of the plurality of beads and is sized to enable the beads seat within and block the larger filter holes thereby decreasing flow of the liquid through the larger filter holes; wherein each smaller filter hole has a diameter that is smaller than the diameter of the larger filter holes and sufficiently smaller than the smallest outer diameter of the plurality of beads such that the beads cannot block the flow of the liquid through the smaller filter holes; and wherein blocking of the larger filter holes by one bead automatically prevents any additional bead from entering the microwell because of a decreased flow rate of the liquid through the microwell, while the smaller filter holes enable the liquid to drain slowly from the microwell to relieve pressure and to inhibit the beads from unblocking the one or more larger filter holes.

In another aspect, the disclosure provides methods of selectively trapping targets in one or more microwells of interest on a microwell array. The methods include identifying one or more microwells of interest; and selectively exposing the one or more microwells of interest to light to induce polymerization of a polymer solution in the one or more microwells of interest, thereby trapping targets in the one or more microwells of interest.

In some embodiments, identifying one or more microwells of interest includes analyzing florescent signals from the microwell array. In some embodiments, the one or more microwells of interest are exposed to light using a photomask. In certain embodiments, the one or more microwells of interest are exposed to light using a projector. In some embodiments, the targets are beads, nucleic acid constructs, or proteins.

In another aspect, the disclosure provides methods of selectively releasing targets in one or more microwells of interest on a microwell array. The methods include identifying one or more microwells of interest; selectively exposing the microwells on the array to light except the one or more microwells of interest, wherein targets in microwells on the array except the one or more microwells of interest are trapped in the microwells due to polymerization of a polymer solution; and collecting targets from the one or more microwells of interest.

In some embodiments, identifying one or more microwells of interest comprises analyzing florescent signals from the microwell array. In some embodiments, the one or more microwells of interest are exposed to light using a photomask. In some embodiments, the one or more microwells of interest are exposed to light using a projector. In some embodiments, the targets are beads, nucleic acid constructs, or proteins.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a diagram comparing a flowchart of a common protein bio-discovery method to a flowchart of an embodiment of the next generation protein discovery methods described in the present disclosure.

FIG. 1B is a schematic diagram showing a general flowchart of an embodiment of the ultra-high throughput protein discovery methods described in the present disclosure.

FIG. 1C is a schematic diagram of cross-section of a pair of microwells as described herein, including a bead that is larger than filter holes at the bottom of the microwell.

FIG. 1D is a schematic diagram of cross-section of a pair of microwells showing one embodiment of the geometry of the microwells and their surface modifications.

FIG. 1E is a schematic diagram of a top-view of two different microwell arrays, each showing a different microwell array design.

FIG. 1F is a schematic diagram of top-view (left) and cross-section (right) of different embodiments of filter hole patterns and designs.

FIG. 2A is a schematic diagram showing methods of adding a reagent into the microwell by dipping the array upside-down into a reservoir. Liquid can fill into the microwells through the top (now bottom) opening via capillary force. The microwell arrays can also be dipped right side up, and then the liquid would enter via the one or more filter holes.

FIG. 2B is a schematic diagram showing that beads can be added into the microwells, e.g., flipped upside-down as shown. The filter holes can block and retain the beads.

FIG. 2C is a schematic diagram showing a system that can add a small amount of reagents to the microwells through the one or more filter holes via a microfluidic channel. The reagents are added to the microwells using capillary force, pressure, or a vacuum.

FIG. 2D is a schematic diagram showing a system that can add reagents through the microwell filter holes from a microarray of reagent droplets, wherein the reagent enters the microwell by capillary force.

FIG. 2E is a schematic diagram showing a fluid jetting system that adds reagents to the microwells through the top opening.

FIG. 2F is a schematic diagram showing a system of filtering and/or washing beads by using pressure or vacuum, with a waste reservoir arranged adjacent to the filter holes.

FIG. 2G is a schematic diagram showing a microwell array system in which a plate is used as a cover to seal the filter holes so that the microwells can be used as regular containers.

FIG. 2H is a schematic diagram showing a microwell array system in which a first plate is used as a cover to seal the filter holes and a second plate is used as a second cover to seal the top openings of the microwells.

FIG. 2I is a schematic diagram showing an embodiment in which the microwell array is used to deposit, e.g., by stamping, liquid contents from inside the microwell onto a substrate to create an array of deposits, wherein each location has a specific coordinate that corresponds to a specific microwell in the microwell array.

FIG. 2J is a schematic diagram showing an embodiment in which the microwell array is used upside-down over a microarray of components (e.g., oligonucleotides) to perform reactions inside the microwells. A plate is used as a cover to seal the filter holes at the “top” of the microwells in this embodiment.

FIG. 2K is a schematic diagram showing an embodiment in which the microwell array is sealed at its upper side to a bead array with oligonucleotides to perform reactions inside the microwells, which contain beads, and includes a plate used as a cover to seal the filter holes.

FIG. 3 is a schematic flow chart that shows how a microwell array as described herein can be used to as a reaction vessel to perform gene synthesis.

FIG. 4 is a schematic flow chart that shows how a microwell array as described herein can be used to amplify a single copy of DNA through PCR or isothermal amplification method. Cell-free protein synthesis reagent can be added into the microwells to produce proteins through in vitro transcription translation (IVTT). Target proteins generated during IVTT can be captured by protein binding beads through affinity binding.

FIG. 5 is a schematic flow chart that shows how one or more liquids containing nucleic acid constructs are transferred to a surface or a blotting plate to form an array of deposits. Protein bound on the beads are washed and assayed to measure the function of the protein. The array of deposits can be decoded to determine the sequences of containing nucleic acid constructs on the array. Alternatively, location information from the microwells used in a protein function assay can be used to release nucleic acid constructs selectively for sequencing.

FIG. 6A is a series of schematic diagrams that shows an example of a fabrication method to make the microwells with filter holes as described herein using a Silicon-On-Insulator (SOI) substrate.

FIG. 6B is a series of schematic diagrams that shows an example of a fabrication method to make the microwells with filter holes as described herein using standard photolithography on a bare silicon wafer.

FIGS. 6C and 6D are a series of schematic diagrams that show an example of microwell array design (6C) and a fabrication method (6D) to make “funnel” shaped microwells as described herein using anisotropic wet etching of silicon wafer.

FIGS. 6E and 6F are a series of schematic diagrams that show an example of microwell array design (6E) and a fabrication method (6F) to make “wine-glass” shaped microwells as described herein using anisotropic plasma etching of silicon wafer.

FIG. 6G is a series of schematic diagrams that shows an example of a fabrication method to make the microwells with filter holes as described herein using a silicon substrate with silicon dioxide or silicon nitride deposited on the surface.

FIG. 7A is a series of schematic diagrams that illustrate how the outside and inside surfaces of the microwells as described herein are modified differently.

FIG. 7B is a series of schematic diagrams that illustrate how certain areas of the outside surfaces of the microwells as described herein can be modified.

FIG. 8 is a schematic diagram illustrating an embodiment of a system to carry out a low dead-volume method to load a liquid reagent into microwells by spin-coating. The liquid reagent is added to the microwell arrays and is spread out by controlling the rotation rate of the microwell array. The liquid fills into microwells by forces caused by the rotation, as well as by one or more of capillary force, pressure, or vacuum.

FIG. 9A is a schematic diagram illustrating an embodiment of a centrifuge system to transfer liquid inside microwells out through the filter holes using centrifugal force, e.g., to a blotting plate. This operation and system can also be used to filter/wash beads.

FIG. 9B is an example of a fluorescence image of a blotting plate, which shows that Green Fluorescent Protein (GFP) in the microwells has been transferred to the blotting plate using an embodiment illustrated in FIG. 9A.

FIG. 10 is a schematic diagram illustrating an embodiment of a system to carry out a method using light to trap nucleic acid constructs (e.g., DNA) by cross-linking a photosensitive polymer with nucleic acid constructs on a surface, e.g., a blotting plate.

FIG. 11 is a schematic diagram illustrating an embodiment of a system that can selectively capture and/or release nucleic acid constructs (e.g., DNA) using light, wherein light causes a charge switch from positive to negative, or vice versa, on a surface, e.g., a blotting plate.

FIG. 12A is a schematic diagram of an example of a system that analyzes microwell images, generates a computer file, prints a specific mask, and traps target contents through photo-polymerization.

FIG. 12B is a fluorescence image of a microwell array showing that GFP proteins in certain microwells have been successfully trapped by a photo-polymerized polymer in those microwells. GFP in other microwells has been washed away.

FIG. 12C is a schematic diagram of an example of a fully automated system that images microwells, analyzes images by computer, generates a specific light pattern through a projector, and traps target content through photo-polymerization.

FIG. 13A is a schematic overview of a coupled expression and CRISPR-Cas nuclease assay from a single DNA fragment, performed in a single reaction chamber, as enabled by IVTT. The schematic shows the reaction and the elements present on the single DNA fragment.

FIG. 13B is an image of a gel that shows a time course of reaction activity. Arrows describe the DNA fragments, with the presence of the cleaved short DNA fragment at all time points after 30 minutes indicating successful expression, reconstitution, and cleavage activity of the CRISPR-SpCas9 ribonucleoprotein complex.

FIG. 14A is a fluorescence image demonstrating that GFP has been expressed inside certain microwells in the array through in vitro transcription and translation (IVTT) by using the PURExpress® system.

FIG. 14B is an enlarged image of FIG. 14A showing that microwells containing beads have elevated GFP signal. The beads have been modified to have GFP genes on the surface. The amount of GFP expression is correlated with the number of beads in the microwells.

### DETAILED DESCRIPTION

The present disclosure relates to methods and systems for searching for proteins having various functions from millions of different organisms and for engineering these proteins for different purposes. Examples include antibody, single-chain antibody, ligases, transposases, methylases, nucleases, transcription factors, sortase, kinases, ubiquitinases, adenylases, proteases, phosphatases, deubiquitinases, anti-microbial peptides, defensin, receptor-interacting peptides/protein. In other examples, proteins can be one or more components of a Clustered Regularly Interspaced Short Palindromic Repeats (“CRISPR”) system.

The ultrahigh throughput protein discovery systems described herein allow one to access untapped resources of biodiversity. As compared to some traditional approaches, the ultrahigh throughput protein discovery described herein is based on genomic and metagenomic mining of many living organisms. After candidate sequences are identified, variations (e.g., random mutations or designed mutations) are introduced into the candidate sequences, and an ultrahigh throughput method is used to screen these proteins for specific, e.g., desired, functions. This approach can dramatically increase the efficiency of finding genes of interest, screening proteins for the desired function, and producing engineered proteins with desired characteristics. The methods described herein represent an ultrahigh throughput-screening tool, and can be used to develop, for example, gene therapies, diagnostic tools, and industrial catalysts, and can also be used in various fields, e.g., medicine, agriculture, and synthetic biology.

There are several key features to the methods and systems described herein for ultrahigh throughput protein discovery: 1) an in vitro synthetic pathway from DNA to RNA to protein and then to the final assay, all completely free of the environmental or cellular context of the original genetic material, providing a high level of control and additional freedom from toxicity; 2) the design and usage of the advanced microwell arrays that enable significantly greater versatility in reagent handling and reactions tested; 3) assay conditions and readouts that are consistent with the required scale, efficiency, and format; and 4) selection methods that enable efficient identification of specific constructs giving rise to positive signal from within a large number of reactions. The successful construction and implementation of the complete system requires a synergistic design that requires innovations on each of the key individual features, as well as how to combine them into efficient methods of ultrahigh throughput protein discovery. An overview of these key features and their integration is provided below.

### 1) Synthetic Screening

Once the natural or engineered protein sequences to be screened are determined, the DNA sequences coding for them are codon optimized and synthesized. This synthetic approach takes advantage of the rapid advances in DNA synthesis capabilities that have yielded increased lengths of high fidelity synthesis products at continually decreasing costs. This differs from past methods of biodiscovery or bioprospecting that have relied on harvesting and amplifying nucleic acids directly from environmental samples (see, e.g., WO1998058085) or required deciphering of specific growth conditions for organisms of interest, many of which were unable to be cultured in a laboratory. Additionally, the synthetic approach allows other functionalization and modification of DNA, including, but not limited to, nucleic acid modifications such as biotinylation, fluorescence tagging, alternative base chemistries such as dideoxy or phosphorothioate modifications for resistance to specific enzymatic activities, and sequence additions such as specific barcodes, hybridization sites, or expression elements such as promoters. Together, the synthetic approach starting at the DNA provides a much more versatile set of methods that can be leveraged for efficient processing and larger scale.

In some embodiments, the synthetic DNA is modified with either barcodes, biotin, or other tags to enable them to be efficiently loaded into microwells. This loading can be enabled either by direct attachment of the DNA to a functionalized surface of the microwells, or via an indirect mechanism in which the synthetic DNA are first loaded onto carriers such as microbeads, which are then deposited into a microwell array for downstream reactions.

After loading the synthesized DNA into the microwell array, the synthetic methods are used to generate the functional RNA and protein macromolecules. In some embodiments, cell free in vitro transcription and translation (IVTT) systems are used, enabling the expression of RNA and protein without any environmental or cellular constraints. This technique differs from traditional bio-discovery approaches in that the methods described herein do not require culturing specific organisms for obtaining bioactive compounds. Thus, the proteins are not subject to culture, toxicity, or other conditions that would need to be either laboriously optimized for individual proteins of interest or otherwise precluded from being screened in cells altogether. Additionally, the synthesis is rapid, yielding amounts of protein compatible with microwell-based reactions in a few hours, versus the days required for traditional methods of recombinant protein expression and purification. Together, these properties enable the use of cell-free synthesis described in these methods to provide greater versatility as the basis for the ultrahigh throughput protein screening platform.

### 2) Microwell Array Design and Usage

The microwell array systems described herein are particularly well suited to enabling such a cell-free, synthetic approach to biodiscovery. Microwells are characterized by their capability to perform a large number of low-volume reactions simultaneously and their reaction versatility. We describe in this system a series of liquid handling operations and instrumentation modifications to perform biological and biochemical reactions in microwell arrays while limiting the waste of reagents and/or samples that are typically costly and/or in very limited supply. We also describe embodiments in which beads-based filtering and washing take advantage of workflows for high throughput macromolecule manipulation and combine them with novel microwell designs to enable greater versatility in reaction conditions. Together, these provide novel capabilities of both throughput and functional diversity to enable ultrahigh throughput protein screening.

In certain embodiments of the invention, beads are used to load the synthesized nucleic acids into the advanced microwell arrays described herein. While beads have been used to separate desired reaction products from byproducts, buffers, and impurities, it is particularly difficult to handle beads in a microwell array screening system. The new microwell array systems described herein feature one or more filter holes at the bottom of the microwells, which can be optionally sealed by a movable plate or equivalent sealing mechanism, such as an oil sealing method. With this design, the microwell array systems can retain the reaction products (e.g., by attaching the reaction products to the beads or functionalized surfaces of the microwells) while other contents in the microwells are removed and/or exchanged. When one or both sides of the microwell array are sealed, the microwell array can also be used as standard vessels or containers for various reactions. The systems described herein allow numerous reactions (e.g., DNA synthesis, transcription, translation, and function assays) to occur in a single microwell platform, which makes large-scale biodiscovery (including e.g., gene synthesis, non-cellular protein synthesis, and screening assays) possible.

### 3) Assays

An ultrahigh throughput biodiscovery platform requires the ability to support versatile reactions and assays to assess function across a wide range of protein activities. Past discovery platforms based on low volume techniques such as microwells or microfluidics can be limited in their ability to facilitate exchange of reaction environments such as buffers, ion concentrations, substrates, and pH. In the system and methods described herein utilizing in vitro transcription and translation reagents (IVTT), the proteins of interest synthesized can be separated from the IVTT mixture, enabling a wide range of functional assays that may not otherwise be compatible with the IVTT mixture.

Additionally, we describe assays formats that can be adapted for this system so that they can be read out in parallel and high throughput. In some embodiments, these methods include the use of reactions whose functional endpoint results in a fluorescent signal to enable rapid detection via microscopy. In other embodiments, the functional endpoint results in the generation or release of a nucleic acid barcode fragment or similar identifier that can be collected and sequenced to identify the proteins resulting in positive functional activity. Altogether, the ability to exchange the solutions in the reaction environment expands the range of assays that can be performed in low volume microwells, generalizing them to encompass more conventional macro-scale biochemical or molecular biology workflows that would be challenging and costly to scale.

### 4) Identification and Selection

The new systems are capable of assaying large numbers of proteins and reactions in a given run, and so one challenge is the specific identification and retrieval of the candidates that yielded the positive result. In bead or droplet-based systems, sorting has conventionally been used to enrich the samples that have a positive signal. In a microwell-based platform in which the reactions are not assayed sequentially and are instead distributed spatially, alternate methods of extracting the identity from positive signal are needed.

Given that the ability to identify specific constructs that provide a positive signal from a large number of reactions is a critical component of efficient protein discovery and engineering, several technologies that are compatible with the system and methods are described herein. These range from direct selection of the positive wells, to targeting constructs using barcodes onto a pre-labeled array, to decoding a randomized array, to other molecular biology reactions that enable the release of signals that enable the retrieval of the exact constructs that led to positive activity.

By combining and analyzing these two streams of information of the positive signal as well as the underlying sequence, the sequence-to-function information for a large number of genes can be obtained. In some embodiments, the reaction is run and the DNA sequence is analyzed in the same microwell.

### Ultrahigh Throughput Protein Discovery Overview

The present disclosure provides ultrahigh throughput screening methods and platforms characterized by great versatility and ultralow volume of reaction reagents.

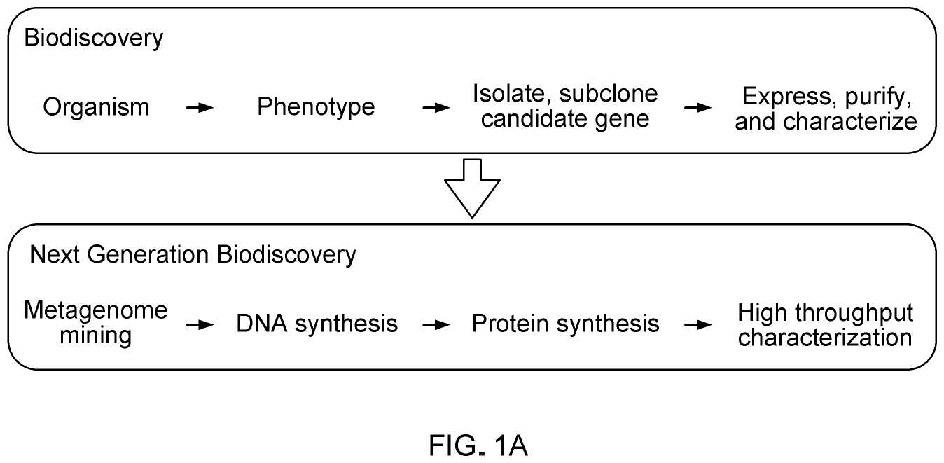

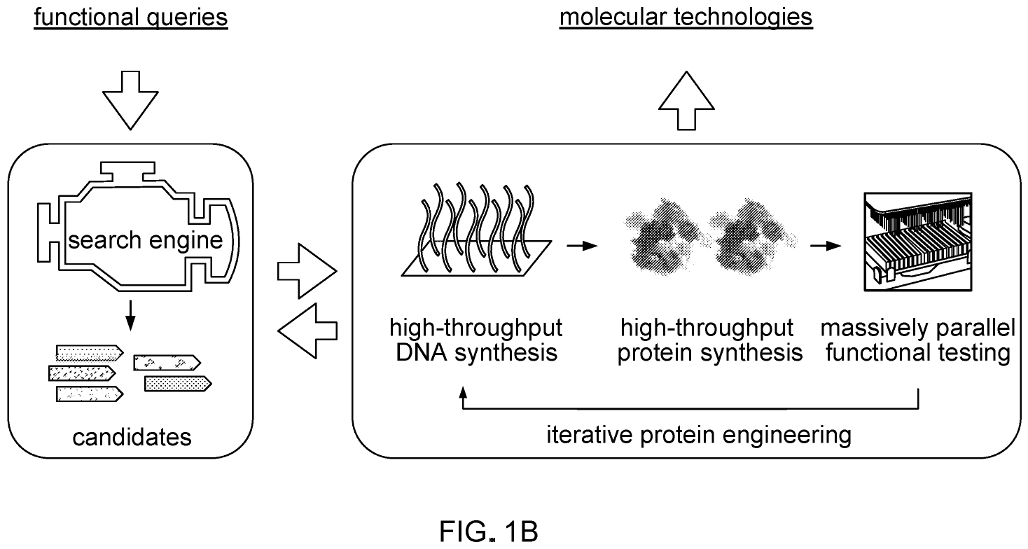

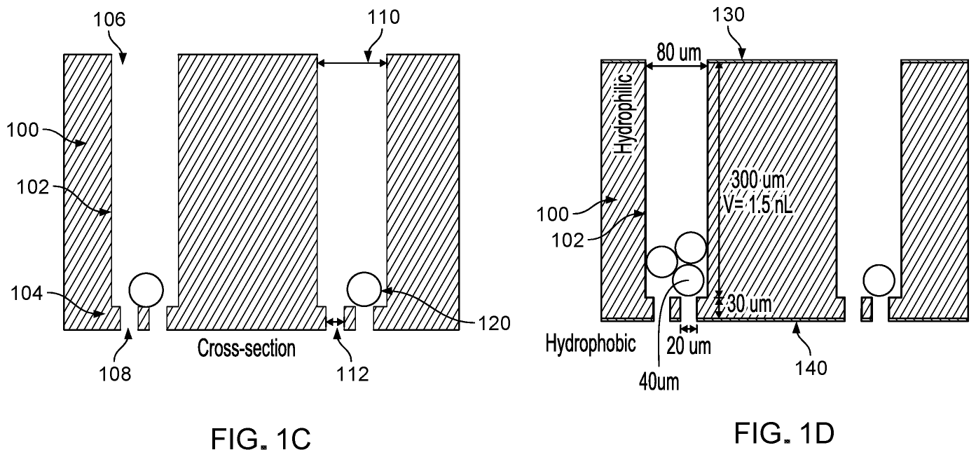

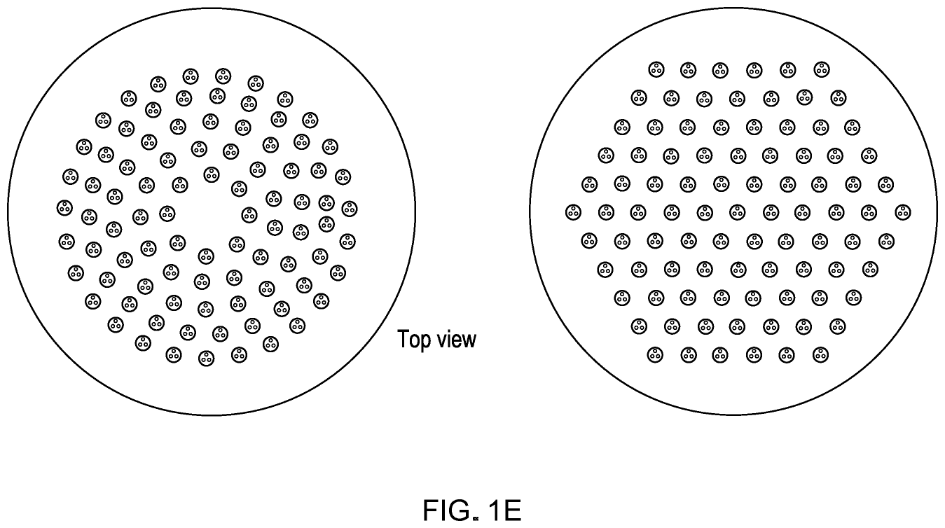

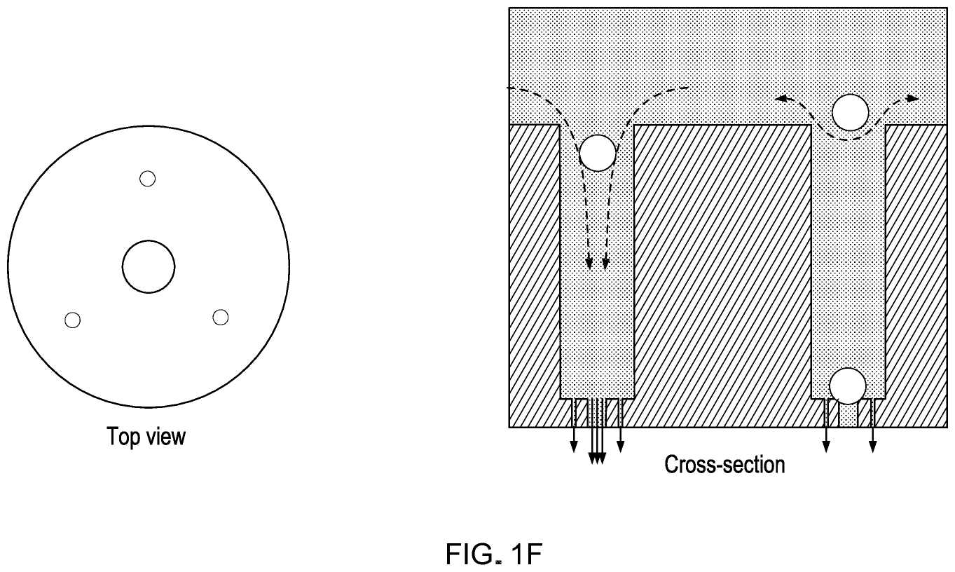

FIG. 1A compares a traditional bio-discovery method with an embodiment of the ultra-high throughput protein discovery methods described herein. As shown in FIG. 1A and FIG. 1B, candidate nucleic acid sequences are first identified by so-called “metagenome mining” by performing functional queries in nucleic acid databases (e.g., databases that have DNA and/or RNA, sequence information of 100, 1K, 10K, 100K, 1M, 10 M, 100 M, 1 B or even more different naturally occurring organisms and metagenomic samples) to find candidate sequences that are expected to encode proteins that may have a specific desired function. Nucleic acid sequence with in silico “mutations” (with either naturally occurring or manmade alterations) can also be prepared and screened.

In a first general step, the systems can synthesize physical nucleic acid constructs (e.g., DNA and/or RNA) in a single linear or circular form. As used herein, the term “nucleic acid construct” refers to a DNA, RNA, or other nucleic acid molecule. Such nucleic acid molecules are synthetic, but can be or include naturally occurring sequences and/or manmade sequences.

The nucleic acid constructs encode either the active components (e.g., enzymes or ribozymes) or substrates of a reaction. In some embodiments, the nucleic acid constructs encode the active protein components (e.g., an enzyme, a ribozyme, or one or more components of a CRISPR system) of a reaction of interest. The active components can then act on a chemical or a biological substrate. In some embodiments, the nucleic acid constructs encode the substrate of a reaction (e.g., a ligand, or a protein that can be modified (e.g., phosphorylated) by an enzyme). The substrates can be modified or catalyzed by the active components in a reaction.

In a next step, the systems automatically dispense the synthesized nucleic acid constructs (e.g., DNA or RNA) into containers, e.g., microwells, droplets, or beads. In some embodiments, one copy or multiple copies of a single nucleic acid construct are dispensed into a single microwell or droplet, or a single copy or a few copies are dispensed and amplified within each microwell. In other embodiments, multiple constructs can be loaded onto a single microwell, droplet, or bead to enable expression of one or more constructs simultaneously. These microwells or droplets have a very low volume, and can range from about 0.5 picoliters to about 50 nanoliters.

In the next step, in vitro transcription and translation (IVTT) reagents are automatically added to the microwells or droplets to perform high throughput protein synthesis, which results in expression of RNA and protein products from the nucleic acid constructs. Then, the systems can automatically add to the microwells or droplets active reaction reagents and/or substrates that are common to all reactions. These materials can be introduced before, simultaneously, or after the addition of IVTT reagents.

In the next step, the systems can incubate the RNA products (e.g., ribozymes, noncoding RNAs) and/or protein products generated from in vitro transcription and translation with the reaction reagents and/or substrates for a period of time (e.g., at least 5 minutes, 10 minutes, 15 minutes, 20 minutes, 30 minutes, 1 hour, 2 hours, 3 hours, 4 hours, or more) at a certain temperature (e.g., 25° C., 37° C., or other temperatures), sufficient to produce reaction products that can be detected and/or measured using massively parallel functional testing assays. Various known detection methods can be used, including, spectroscopy (such as fluorescence spectroscopy, ultraviolet-visible spectroscopy (UV-VIS), Raman spectroscopy, surface enhance Raman spectroscopy, and absorption spectroscopy), spectrometry (e.g., fluorometry), surface plasmon resonance, field effect transistor, and second-harmonic generation. In addition to the various assay techniques, other methods can be used to capture or release specifically constructs that demonstrate the desired activity for identification. Based on the detection results, the systems automatically provide the user with a functional characterization report for the RNA products and/or protein products in each separate well or droplet, whose location within a coordinate system is known. Then the systems automatically determine and identify the nucleotide sequence information of the nucleic acid constructs that correspond to a specific reaction result.

Proteins with desired characteristics can be selected for further genome mining and engineering. FIG. 1B shows a more detailed schematic than what is shown in FIG. 1A, and includes a feedback loop of iterative protein engineering in which the sequences of proteins identified to have desired characteristics are used for another round of high throughput DNA synthesis with some variations (e.g., mutations). This automated iteration process can generate more candidate sequences for screening. In some embodiments, the proteins can be further engineered, which can further improve the desired characteristics. This process can be repeated for a sufficient number of times, until proteins with the desired characteristics are generated.

The systems described herein also provide a versatile platform for a variety of different assays. In these platforms, multiple assays can be developed for different protein activities. These activities include, e.g., enzymatic activity, binding activity, cleavage activity, and bond-formation. In some embodiments, these activities generate optical signals (e.g., fluorescence, chemiluminescence, phosphorescence, color change, absorption change, and precipitation) or non-optical signals (e.g., heat, pH, volume, capacitance, impedance, conductivity, and other physical change), which can be detected by appropriate devices. In some embodiments, one single assay can be used to detect high dynamic range for individual activities. In some embodiments, multiple related activities can be screened in a single assay.

### Microwell Arrays

In some embodiments of the system, microwell assays are used to provide a technology that enables high throughput and versatile reaction conditions. The microwell arrays can be used to synthesize and/or screen nucleic acid constructs, peptides, and proteins. The advanced microwell designs used in the microwell arrays described herein can serve as the container for low volume reactions (FIG. 1C) and have specific features that enable them to be used in more adaptable ways than in prior arrays. Additionally, the low volume of microwell reactions can greatly improve screening efficiency and, in the meantime reduce the cost of individual reactions.

As shown in FIG. 1C, each microwell (100) having sidewalls (102), a bottom (104), and a top opening (106). Each microwell has one or more, e.g., 2, 3, or 4, filter holes (108) arranged at the bottom (104) of the microwell. In some embodiments, the volume of each isolated microwell is from about 0.5 picoliters (pL) to about 50 nanoliters (nL), e.g., from 4 pL to 1 nL, from 4 pL to about 500 pL, or from 100 pL to 500 pL. In some embodiments, the volume of the isolated microwell is less than 50 nL, 10 nL, 5 nL, 1 nL, 900 pL, 800 pL, 700 pL, 600 pL, 500 pL, 400 pL, 300 pL, 200 pL, 100 pL, 50 pL, or 10 pL. In some embodiments, the volume of the isolated microwell is greater than 1 pL, 4 pL, 5 pL, 10 pL, 100 pL, 200 pL, 300 pL, 400 pL, 500 pL, 600 pL, 700 pL, 800 pL, 900 pL, 1 nL, 5 nL, or 10 nL.

Each microwell can have a diameter (110) of from about 10 to about 100 microns (μm), e.g., from 20 to 100 μm, from 30 to 90 μm, or from 60 to 80 μm. In some embodiments, the diameter (110) is less than 100 μm, 90 μm, 80 μm, 70 μm, 60 μm, 50 μm, 40 μm, 30 μm, 20 μm, or 10 μm. In some embodiments, the diameter (110) is greater than 5 μm, 10 μm, 20 μm, 30 μm, 40 μm, 50 μm, 60 μm, 70 μm, 80 μm, 90 μm, or 100 μm.

In some embodiments, the filter holes (108) have a diameter (112) of from about 0.5 to 40.0 μm, e.g., from 1 to 40 μm, from 1 to 30 μm, from 5 to 20 μm, or from 10 to 20 μm, In some embodiments, the diameter (112) is less than 40 μm, 30 μm, 20 μm, 10 μm, 5 μm, or 1 μm. In some embodiments, the diameter (112) is greater than 0.5 μm, 1 μm, 2 μm, 3 μm, 4 μm, 5 μm, 10 μm, 20 μm, 30 μm, or 35 μm.

In some embodiments, as shown in FIG. 1F, filter holes have two or more different sizes. A larger filter hole is somewhat smaller than the smallest outer diameter of the plurality of beads used with the system. When a bead enters a microwell driven by hydrodynamic flow, capillary force, and/or a differential pressure, the flow of the liquid will focus the bead towards the one larger filter hole. The bead is then seated snugly into the hole, but does not pass through the larger filter hole and thus blocks liquid passing through the filer hole. Liquid will pass through one or more smaller filter holes in a much smaller flux rate. The decrease in flux rate will automatically prohibit or reduce the probability of other beads entering the microwell that already has been loaded with a bead. This self-limiting loading method will load one bead, and no more than one bead, into more microwells than predicted by the Poisson distribution (i.e., super-Poisson loading). There will thus be fewer empty microwells with no beads, and far more microwells that contain one bead compared to a predicted Poisson distribution.

In some embodiments, a larger filter hole is slightly smaller than the size, e.g., maximum outer diameter, of the beads, and the smaller filter holes are much smaller than the size of the beads. When a bead is loaded into the microwell, it will block the large filter hole and significantly decrease the rate of flow through the microwells, which prevents other beads from entering into this microwell. Such a self-limiting loading method could achieve higher bead loading ratios than regular Poisson loading (i.e., super-Poisson loading). In other words, many more microwells will contain only one bead than would be predicted by normal Poisson statistics. According to Poisson statistics, when the average rate of occurrence equals 1 (e.g., the number of beads equal the number of wells), the probability of microwell with a single bead is only 37%, while 26% of microwells will have two or more beads. This is why the average rate of occurrence should be kept low, e.g., <0.3, to minimize the chance of having two or more beads in a well. The drawback is that majority of microwells are empty (e.g., 74% of microwells are empty when the average rate of occurrence is 0.3). Self-limiting loading methods allow using higher values of the average rate of occurrence, but limit the ratio of loading multiple beads into a microwell.

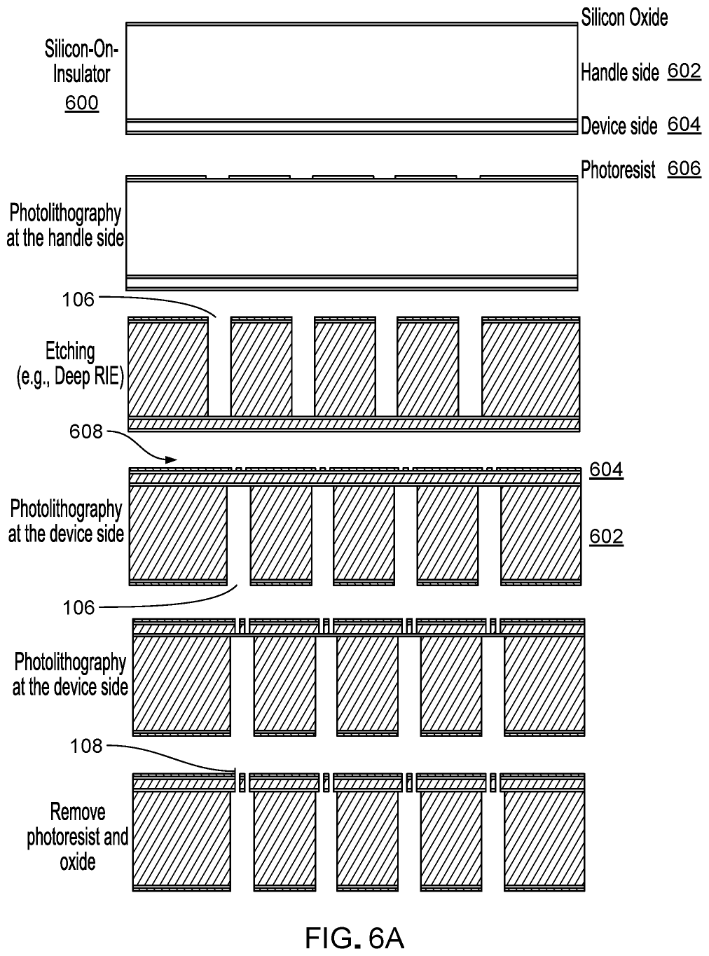

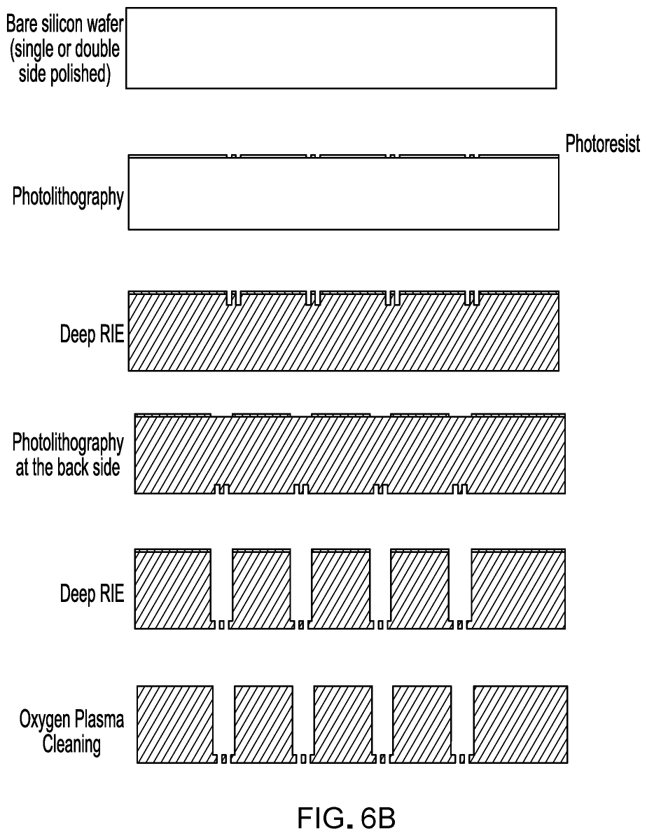

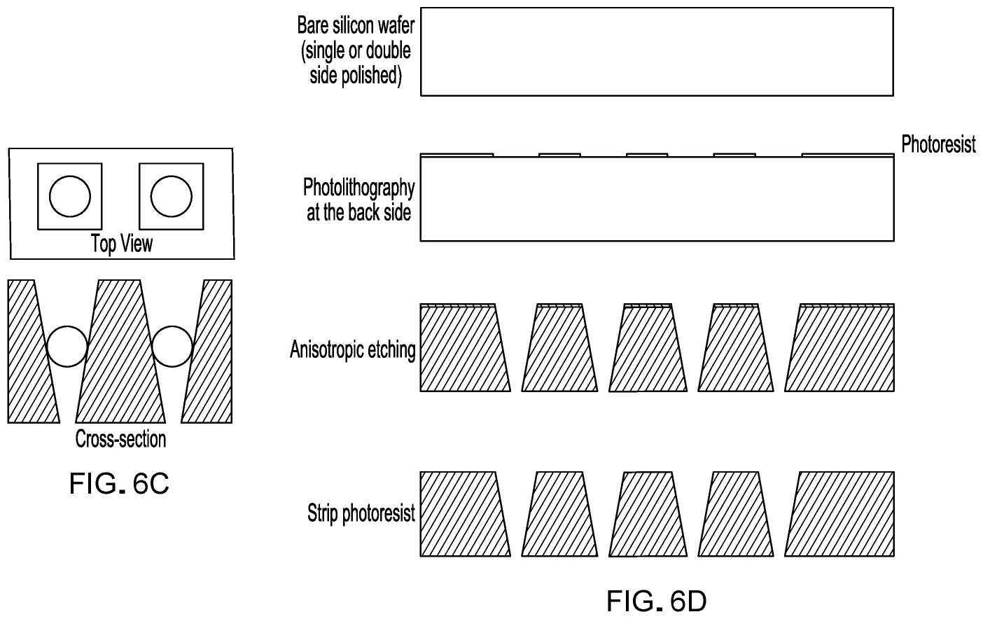

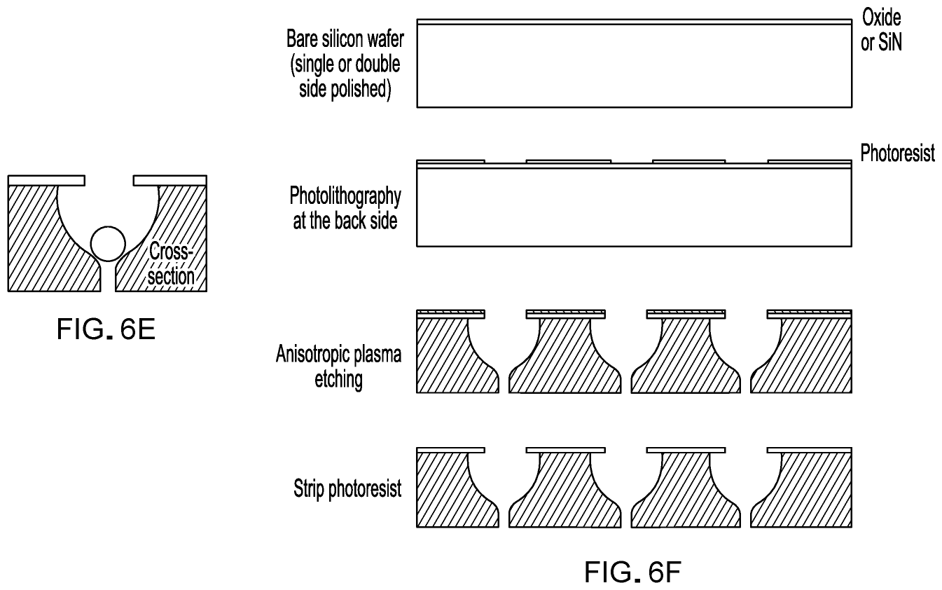

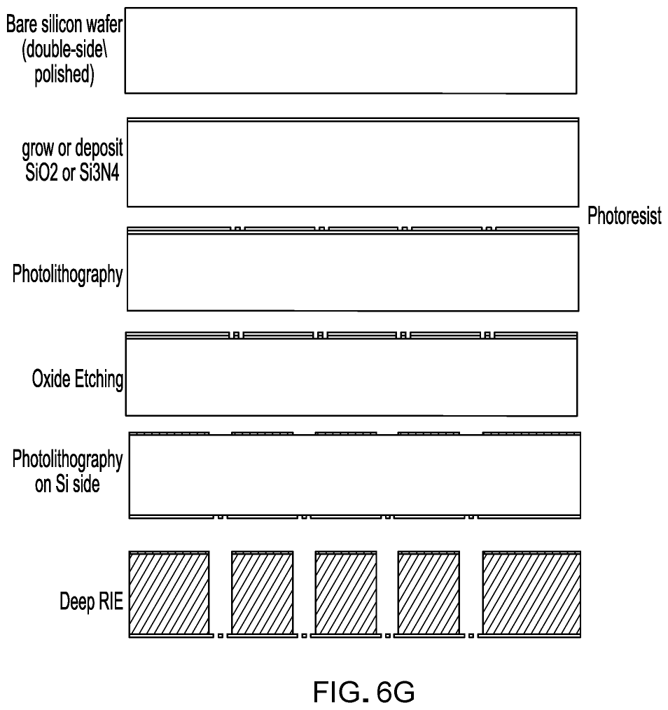

In some embodiments, microwells and/or filter holes (e.g., FIG. 6C, top view) have shapes other than a circle. For example, rectangular microwells having four corners are beneficial to hold liquid to slow down evaporation.

In some embodiments, microwells and filter holes are blended together, e.g., “funnel” shaped (FIG. 6C) and “wine-glass” shaped (FIG. 6E). These shapes have an advantage in that they can be fabricated in a single etching step using anisotropic wet (FIG. 6D) or dry (FIG. 6F) etching.

In some embodiments, the opening at one end is bigger than the opening at another end. The beads can enter into the microwells through the bigger opening, but cannot exit the microwells through the smaller opening.

The microwell arrays can be used to screen nucleic acid constructs, peptides, and proteins, e.g., enzymes, for specific functional activities at ultra-high throughput. FIG. 1E showed two embodiments of the array design. In some embodiments, there are more than 1,000 microwells, more than 5,000 microwells, 10,000 microwells, more than 50,000 microwells, 100,000 microwells, 200,000 microwells, 300,000 microwells, 400,000 microwells, 500,000 microwells, 1,000,000 microwells, 2,000,000 microwells, 5,000,000 microwells, 10,000,000 microwells, or more than 20,000,000 microwells on a microwell array. In some embodiments, there are more than 100 microwells, more than 1000 microwells, 2000 microwells, 5000 microwells, 10,000 microwells, 50,000 microwells, 100,000 microwells, or 200,000 microwells per square centimeter.

The microwell arrays can be used with various types of affinity beads (120), including chemical or protein conjugation, nucleic acid hybridization. These beads can have various properties, e.g., non-magnetic or magnetic beads, affinity beads (e.g., beads with chemical or protein conjugate, or with nucleic acids for hybridization), or beads that are detectable via fluorescent or other markers or reporting agents, as described in more detail below. The beads have a diameter that can be greater than the diameter of the filter holes (112) and smaller than the diameter of microwells (110). In some embodiments, the bead diameter is greater than 0.5 μm, 1 μm, 2 μm, 3 μm, 4 μm, 5 μm, 10 μm, 20 μm, 30 μm, or 35 μm. In some embodiments, the diameter of beads is less than 40 μm, 30 μm, 20 μm, 10 μm, 5 μm, or 1 μm, but greater than the diameter of filter holes (112).

These beads can provide a convenient way to separate reaction products (or reaction agents) from other undesired contents (e.g., reaction byproducts). In different embodiments, the reaction agents or reaction products (e.g., nucleic acids, DNA, RNA, oligo nucleotides, proteins, and peptides) can attach to the beads, for example via a functional group, e.g., an antibody or one member of a binding pair, e.g., a chemical or ligand binding pair. Because the beads cannot pass through the filter holes, the reaction agents or reaction products that are attached to the beads will remain in the microwells, and the other agents in the one or more liquids (e.g., buffers, water, reaction byproducts, waste liquid) can be removed, e.g., through the filter holes (108), by various means, e.g., pressure, vacuum, or centrifugal force.

The beads used herein can be fabricated from materials known in the art. Examples of such materials include e.g., inorganics, natural polymers, and synthetic polymers. Examples of these materials include, e.g., cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like, polyacrylamides, latex gels, polystyrene, dextran, rubber, silicon, plastics, nitrocellulose, celluloses, natural sponges, metals, plastics, cross-linked dextrans (e.g., Sephadex™) agarose gel (Sepharose™), or other materials known to those of skill in the art. In some embodiments, the beads can be streptavidin polymer beads, streptavidin-coated magnetic particles (Spherotech, Lake Forest, Ill.), AMpure® beads (Beckman Coulter, Brea, Calif.), Dynabeads® M270 (Thermo Fisher Scientific, Waltham, Mass.), or SPRI® beads (Agencourt AMPure® XP beads, Beckman Coulter, Brea, Calif.; Cat. No. A63881). In some embodiments, the beads are magnetic, paramagnetic, or superparamagnetic beads.

The platforms described herein can be used to image multiple microwells simultaneously and/or individually. In some embodiments, more than 10,000 microwells, more than 50,000 microwells, 100,000 microwells, 200,000 microwells, 300,000 microwells, 400,000 microwells, or 500,000 microwells can be imaged simultaneously.

The systems described herein also isolate each microwell from other microwells, eliminating crosstalk or contamination between microwells. In some embodiments, oil can be used to seal the microwells. In some embodiments, the oil sealing can be maintained for at least 1, 2, 3, 4, 5, 10, 15, 20, 25, 30, 35, 40, 45, 50, or 55 minutes, or for 1, 2, 3, 4, 5, or more hours, e.g., 10, 15, 20, 24 hours, 3 days, or 1 week. In some embodiments, a movable plate can be used to seal one or more of the top openings (106) and/or one or more of the filter holes (108). In some embodiments, the movable plate can have a hydrophobic surface.

The microwell arrays can be made by many methods known in the art, e.g., etching, photodeposition, additive manufacturing (e.g., 3-D printing), photolithography, thin film deposition, UV-LIGA (Lithographie, Galvanoformung, and Abformung) imprinting, injection molding, embossing, particle blasting, and laser cutting.

FIG. 6A illustrates a fabrication method on Silicon-On-Insulator (SOI)substrate, which is one example of a method that can be used to fabricate the microwell arrays described herein. The SOI substrate (a silicon-on-insulator wafer) (600) has a handle side (602) and a device side (604). The thickness of the handle-side (602) can range from 100 μm to 1,200 μm, e.g., from 200 μm to 1,000 μm, from 200 μm to 800 μm, from 200 μm to 600 μm, from 200 μm to 500 μm, from 400 μm to 1,000 μm, or from 600 μm to 1,000 μm. The thickness of device-side (604) can range from 1 μm to 100 μm, e.g., from 10 μm to 100 μm, from 20 μm to 100 μm, from 50 μm to 100 μm, from 1 μm to 50 μm, from 10 μm to 50 μm, or from 1 um to 5 um, 5 um to 10 um. Standard photolithography can be used to coat a light-sensitive photoresist layer (606) on one side of the device (e.g., the handle-side).

At the handle-side, the photoresist layer (606) has a pattern that matches the desired pattern of microwell arrays. The top opening (106) of microwells is not covered by the photoresist layer (606). Thus, the photoresist layer (606) serves a mask and protects the area under the photoresist layer from subsequent etching process. A wet and/or dry etching process can be used to etch silicon oxide and/or silicon layer. An example of a dry etching technique includes deep reactive ion etching (deep RIE) using C₄F₈ and/or SF₆ gas. Since the deep RIE process etches much slower on the silicon oxide layer than on the silicon layer, the etching process can be effectively stopped before reaching the device-side of the SOI wafer, because of the silicon oxide layer between the handle side (602) and the device side (604).

A similar process can be used on the device-side to make the filter holes. The process applies a second photoresist layer (608) to the device side (604) with uncovered areas for filter holes (108). In some embodiments, a mask aligner equipped with an IR light source can perform through-wafer registration using registration markers to fabricate an opening at the locations for filter holes (108). After both microwells and filter holes are fabricated, photoresist and oxide can be stripped away through a standard photolithography procedure. The general methods of making microwell arrays are described in detail, for example, in U.S. Pat. No. 9,409,139B2, and US20160310927A1; each of which is incorporated herein by reference in its entirety.

FIGS. 6B, 6D, and 6F illustrates several fabrication methods using a bare silicon substrate. In FIG. 6B, both sides of the wafers are fabricated and etched using a similar deep RIE as described in FIG. 6A, however the etching depth is controlled by etching parameters (e.g., etching cycles or etching time), instead of using oxide as an etch stop. FIG. 6D illustrates an anisotropic wet etching method (e.g., hydrofluoric acid), which is capable to fabricate a funnel shape microwells in a single step. FIG. 6F illustrates an anisotropic dry etching method to fabricate a wine glass shape microwells in a single step.

FIG. 6G illustrates another fabrication method using a silicon substrate. A layer of silicon dioxide is deposited or grown on one side of the silicon wafer. The silicon dioxide can be etched using a deep oxide etcher. Since the etch rate of silicon is very slow compared to silicon dioxide, the oxide etching process effectively stops at the silicon layer. Similarly, the silicon side can be etched using deep RIE, which will stop at the silicon dioxide layer. Using two substrates that are etching towards a stop plane between them from two different sides simplifies the entire fabrication process. In addition, the silicon dioxide layer provides additional advantages. For example, because the silicon dioxide layer is transparent to the visible light, it allows direct observation of microwells from the filter side. Some other substrates that allow selectively etching from one layer to a silicon layer can be used as well. These substrates include, e.g., silicon nitride, silicon carbide, diamond, and sapphire. These can be deposited or grown on one side of the silicon wafer, and these substrates and the silicon from the silicon wafer can be used as a stop layer for the etching process as described above.

In some other embodiments, a layer of silicon dioxide, silicon nitride, silicon carbide, diamond, or sapphire is deposited or grown on both sides of the silicon wafer. In other embodiments, a substrate that allows selectively etching to silicon (e.g., silicon dioxide, silicon nitride, silicon carbide, diamond, and sapphire) is deposited or grown on one side, and another substrate is deposited or grown on the other side of the silicon wafer.

### Microwell Reagents and Methods of Adding or Removing Reagents

The microwell arrays described herein can allow a series of reactions to occur in the same microwell. These reactions include, e.g., nucleic acid synthesis, nucleic acid assembly, in vitro transcription and translation, and protein functional assays. It is emphasized that these methods for loading reagents can be used to both load the nucleic acid sequences containing the constructs of interest, as well as the reagents needed for their manipulation and reactants and substrates needed for downstream assays. Usually, the reagents for these reactions need to be added to the microwells and once the reactions are completed, the reagents need to be properly removed.

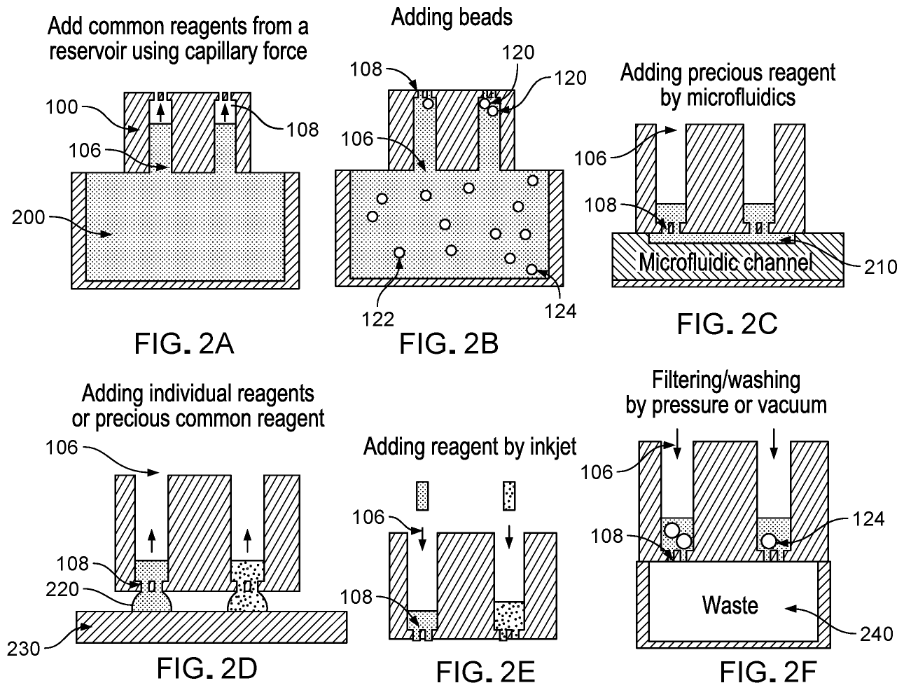

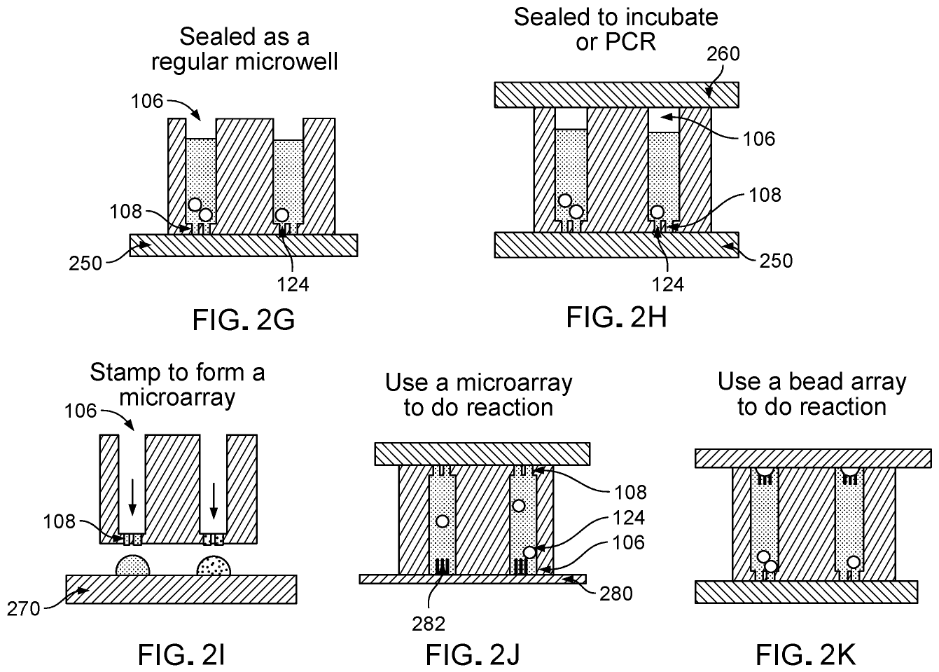

There are many different ways to add liquids (e.g., various reaction reagents) into microwells or remove liquids from the microwells. FIGS. 2A-2K are a series of schematic diagrams of microwells in cross-section that illustrate various methods of handling reagents, including e.g., methods to add reagents into the microwells, methods to remove reagents from the microwells, and methods of using the microwells in different assays.

As shown in FIG. 2A, the microwell array can be placed upside-down, and liquids can be added to the microwells from a reservoir (200) using capillary force through top opening (106) of microwells. Alternatively, a reservoir (200) or O-ring can be placed on the top of the microwells, and liquids can be added to the microwells from the top opening (106) of microwells. Pressure or vacuum can also be used if capillary force is not sufficient to fill the microwells with the liquids.

FIG. 2B shows that beads (120) can be added to the liquids, wherein the beads are suspended in the liquids or trapped in multi-phase emulsion droplets. In this example, the microwell array is positioned upside-down over a reservoir (200) containing a liquid that includes the beads. When liquids fill the microwells, e.g., with capillary force, or a vacuum, or pressure applied to the reservoir, the beads within the liquids are transferred into the microwells at the same time. Because the beads are larger than the filter holes (108), the beads (120) are trapped in the microwells (106) as excess carrier liquid exits through the filter holes, now at the “top” or the microarray as shown in FIG. 2B. In some embodiments, control beads (122) are also added to the liquids. In some embodiments, the control beads do not have a functionalized surface, thus reaction products or reaction agents cannot attach to the control beads. However, the control beads are used to help ensure that certain number of functionalized bead enters each microwell. For example, functionalized beads (124) can be mixed with control beads (122). In some embodiments, when the beads are added to the microwells, the concentration of functionalized beads (124) is selected to be sufficiently small so that at most one functionalized bead (124) is added to one microwell.

FIG. 2C shows a system that automatically adds reagents to the microwells via a microfluidic channel (210) through the filter holes (108). A similar mechanism can also be used to add one or more liquids into the top of the microwells, e.g., by placing a microfluidic channel on top of the microarray, and the one or more liquids can be added through the top opening (106). Furthermore, as both the top and bottom surfaces of the microwell array are flat, both surfaces can be sealed by plates with or without microfluidic channels. In some embodiments, microfluidic channels with pneumatic valves are used to control sophisticated liquid movement. In some embodiments, a microfluidic channel system is placed at the bottom of the microwell array and can be used to add liquids (e.g., reagents) to the microwells through the filter holes. The microfluidic channel system placed at the bottom of the microwell array can also be used for various operations, e.g., removing liquids from microwells, transferring liquids from one microwell to another microwell, or combining liquids in two or more microwells to form an effectively larger reaction vessel.

FIG. 2D shows a system that automatically adds reagents through the filter holes (108) from an array of reagent droplets (220), wherein the reagent enters the microwell by capillary force or by pressure difference. The array of reagent droplets (220) can be prepared by using a fluid jetting system on a flat surface (230), such as glass or silicon. It can also be prepared by using an array of pins to stamp reagents to the flat surface (230). In some other embodiments, the support can be a blotting plate, wherein reagents are not on the surface, but are buried in the matrix of the blotting plate, and the reagents can diffuse into or out one or more of the microwells through the filter holes (108).

FIG. 2E shows a fluid jetting system that can add reagents to the microwells through the top opening (106). The fluid jetting system can add different reagents to different microwells, and can add a particular reagent to selected microwells. In some embodiment, fluid jetting system can add reagents through the filter holes (108).

FIG. 2F shows a system of adding a large amount of one or more liquids to wash beads by applying pressure at the top through the top opening (106) or applying vacuum at the bottom through the filter holes (108). This operation can force one or more waste liquids to enter a waste reservoir (240) or a blotting plate. This method can be used to filter beads, air-dry beads, or empty one or more liquids in the microwells.

FIG. 2G shows how a movable plate (250) can be used to seal the filter holes (108) so that the microwells can be used as regular containers for various reactions. In some embodiments, the movable plate is made of rubber, plastic, glass, or silicon. In some embodiments, the movable plate is not required, because capillary force can hold the liquids in the microwells against gravity.

FIG. 2H shows how a first cover, e.g., a plate or a fluid, e.g., a biocompatible oil, which is immiscible with liquids in the microwell, (250) can be used to seal the filter holes (108) and a second cover, e.g., a plate or oil, can be (260) used to seal the top openings (106) of the microwells. Sealing both sides of the microwells in the array can prevent crosstalk or contamination between microwells, minimize liquid evaporation, and/or allow reactions (e.g., polymerase chain reaction (PCR)) to proceed under appropriate conditions.

FIG. 2I shows an embodiment in which the microwell array is used to deposit, e.g., by stamping, contents in the microwell onto a substrate (270) to create a microarray of deposits, wherein each location has a specific coordinate location that corresponds to a specific microwell in the microwell array.

FIG. 2J shows an embodiment in which the microwell array is used upside-down over a microarray of components (282) (e.g., oligonucleotides) to perform various reactions inside the microwells. In some embodiments, the microwell array is used in its normal position with a microarray of components (282) covered on its top. In some embodiments, components (282) on the microarray can be initially attached on the solid surface (280), and then released into the solution through, e.g., cleavage, restriction enzyme digestion, denature, or charge change, etc.

FIG. 2K is a schematic diagram showing an embodiment in which the microwell array is sealed to a bead array with oligonucleotides to perform reactions inside the microwells.

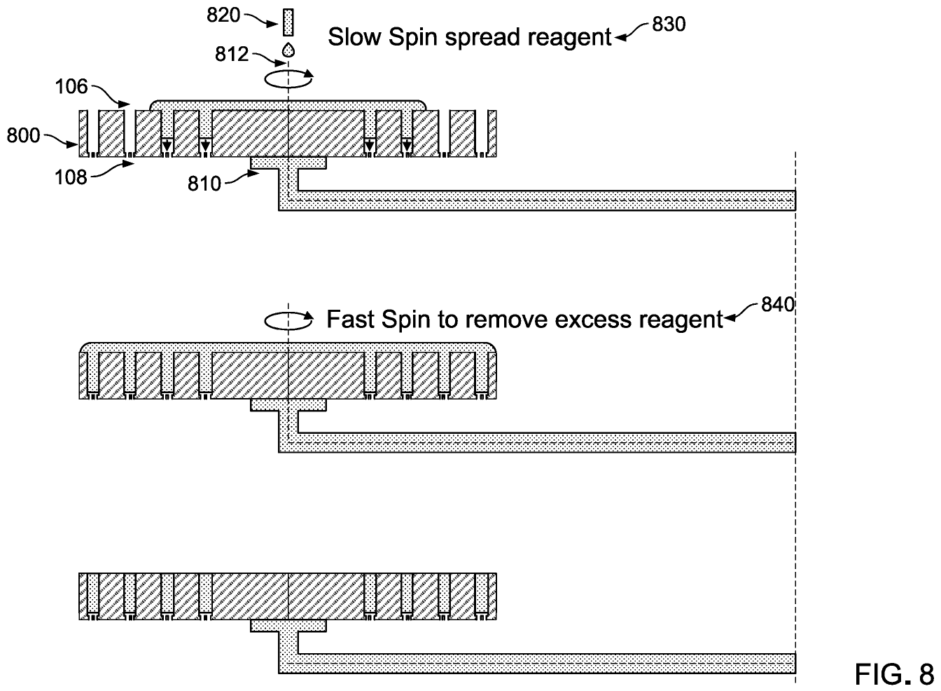

This disclosure also provides various methods to add one or more liquids to the entire microwell array. FIG. 8 shows an embodiment of a system to carry out a low dead-volume method to load one or more liquids, emulsion, or suspension (e.g., reagents) into microwells by spin-coating. A microwell array (800) is placed on a rotating support (810), wherein the support is controlled to rotate around axis (812) using a computer-controlled motor (not shown). Liquids (820) are added onto the microwell array (800) and are spread out by spinning the microwell array (800) at a relatively slow speed (830), e.g., 50, 100, 200, 500, 800, 1000 rotations per minute. Liquids then move outwardly due to centrifugal forces, equally in all directions, and flow into microwells, e.g., by capillary force, pressure, or vacuum. To prevent the loss of liquid to spin-off from the chip, a wall, reservoir, or O-ring can be used to surround the chip during this spreading process.

Excess amounts of the one or more liquids on the top of the microwell array (800) are removed by spinning the microwell array (800) at a relatively high speed (840), e.g., 1000, 1500, 2000, 3000, 4000, 5000, 6000, 7000 rotations per minute. Once the excess amount of liquid is removed, the liquid in each microwell is isolated from the liquid in the other microwells, thus forming individual isolated reaction chambers. In some embodiments, beads are suspended in the liquids either before or after the one or more liquids are added, or before or after a specific liquid is added.

In some embodiments, the chip is placed in a humid chamber to prevent evaporation. In some embodiments, oil (e.g., fluorinated oil, mineral oil, hydrocarbon liquid) is added to cover opening of the microwells to prevent evaporation. In some embodiments, the chip is immersed in immiscible liquid to prevent evaporation.

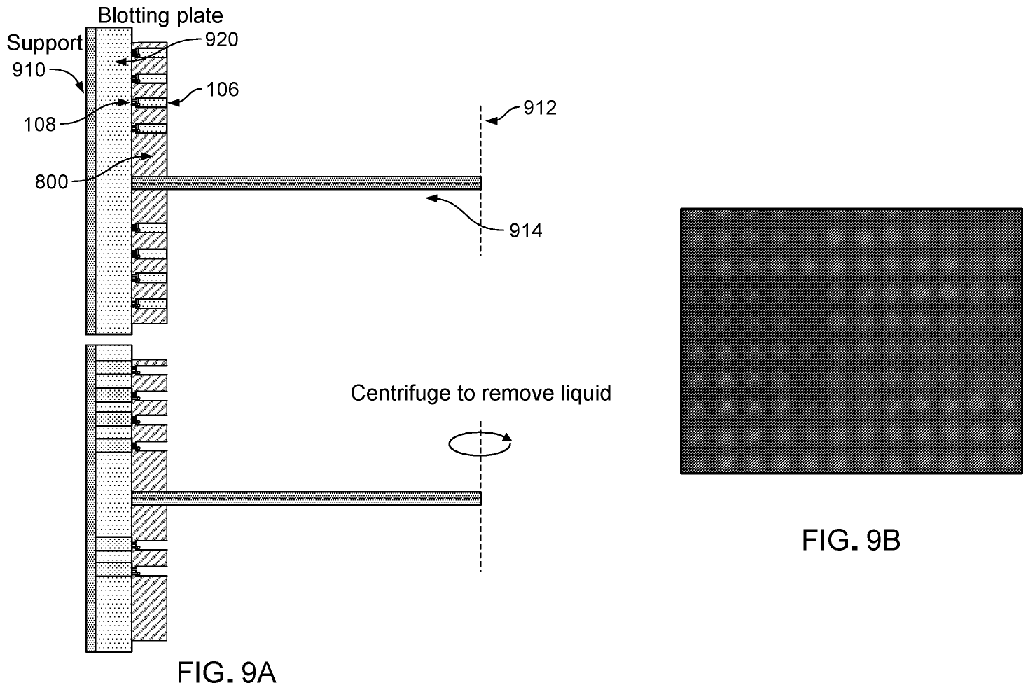

FIG. 9A shows an embodiment of a system to transfer one or more liquids in the microwells out through the filter holes to a waste reservoir or a blotting plate (920) by centrifugation. The microwell array (800) is placed on a support (910), wherein the support is configured to rotate around axis (912), controlled by a computer-controlled motor (not shown). In some embodiments, the support (910) is fixed on a rotation body (914). When the microwell array (800) rotates around the axis (912), the centrifugal force pushes the one or more liquids, emulsion, or suspension out of the microwells onto the blotting plate (920), creating an array of liquid deposits on the plate, wherein each location of the deposit has a specific coordinate that corresponds to a specific microwell in the microwell array. In some embodiment, the blotting plate could be patterned with hydrophobic barriers to limit cross-talk between different location.

FIG. 9B shows the results of an experiment, which demonstrates that proteins (e.g., GFP) can be transferred to a blotting plate (e.g., polyvinylidene difluoride (PVDF) membrane) using the device as shown in FIG. 9A.

In some embodiments, the systems shown in FIG. 8 and FIG. 9A are combined into a single system. For example, the system can have two motors, or one motor geared to control both components of the system. For example, the rotation body (914) can be configured to allow the support (910) to rotate around the direction of the rotation body (914), which is equivalent to the rotation axis (812) in FIG. 8. Thus, the combined system can be used to add one or more liquids, remove liquids, and/or wash beads, etc.

### Nucleic Acid Loading and Polypeptide Synthesis

The systems and methods described herein use a process for producing peptides or peptide derivatives by using a reaction system that transcribes a DNA sequence construct into an RNA and then translating the RNA into a polypeptide. Cell-free protein synthesis is typically simpler than in vivo methods, and requires only the addition of a template DNA or mRNA to a reaction mixture and then incubation for a sufficient time (e.g., several hours) to yield the desired protein. Thus, cell-free protein synthesis provides an effective approach for the high-throughput protein biodiscovery platforms described herein. Moreover, reaction conditions, such as the temperature or accessory factors, can be carefully controlled in cell-free systems.

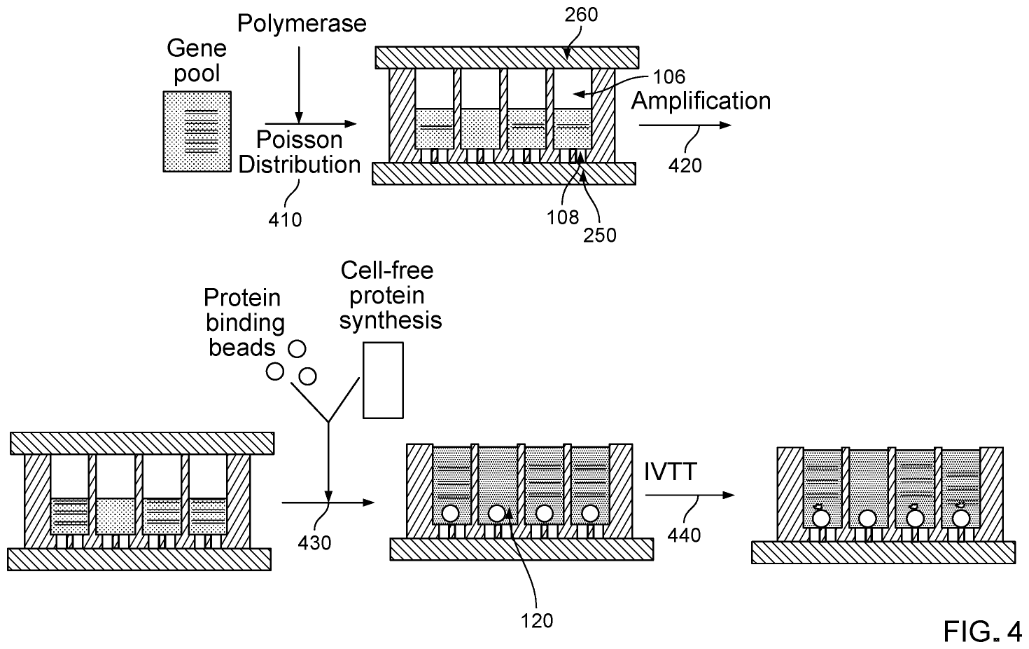

In some embodiments, nucleic acid constructs are directly loaded, e.g., automatically, into a microwell array. For example, nucleic acid constructs can be dispensed from one donor microwell array into an acceptor microwell array that aligns with the wells of the donor array, as displayed in FIG. 4. (410). In one aspect, the microwell array has a functionalized surface on which oligonucleotide binding sequences are covalently attached, facilitating specific directing of nucleic acid constructs to spatial locations in the array by hybridization. In some other embodiments, nucleic acid constructs are synthesized based on the sequences in the gene libraries, and are then bound to affinity beads. The nucleic acid constructs (e.g., DNA or RNA) can be attached to affinity beads by various means. For example, the nucleic acid constructs can have a terminal affinity tag or internal affinity tags. In some embodiments, the affinity tag is biotin, and the beads have immobilized thereto streptavidin, thus, the nucleic acid constructs are attached to these beads through the binding between biotin and streptavidin. Other ligand binding pairs are known in the field and can be used herein. In some other embodiments, the nucleic acid constructs can include an oligonucleotide binding sequence, which can bind to a complementary oligonucleotide binding sequence attached to the beads.

In some embodiments, each bead can have more than one affinity binding molecules (e.g., streptavidin, antibodies, or oligonucleotides). However, the concentration of affinity tagged nucleotide constructs is titrated such that only one or at most one nucleic acid construct is attached or conjugated to each bead. Each bead may have two or more different types of affinity binding molecules attached to its surface. For example, one type of affinity binding can be used to attach target gene, and another type of affinity binding on the beads can be used to capture proteins generated through IVTT.

Beads are distributed across microwell arrays. In some embodiments, each microwell receives one or more beads. In some embodiments, each microwell receives at most one bead. In some embodiments, some or many microwells do not receive any beads. In some embodiments, all microwells receive the same type of bead. In other embodiments, different microwells receive different beads, or groups of microwells receive the same beads, and other groups of microwells receive different beads.

In some embodiments, the beads are not distributed randomly but instead are pre-localized on a bead-array as shown in FIG. 2K. The bead array is sealed with a microwell array of similar spatial distribution and density so that each microwell contains a well-defined set of bead arrays. The bead array contains a gene or a set of genes to be screened. Using the bead array has the advantage in that positions and barcodes can be spatially localized prior to sealing the microwells with the microwell array, so that identification of constructs that led to positive signal in downstream reactions can be more readily achieved. Additionally, a bead array can be loaded at a higher density (e.g., super-Poisson loading) to enable more efficient utilization of the microwell array.

As shown in FIG. 4, a pool of nucleic acid constructs (e.g., gene pool) can be added, along with necessary reagents (such as polymerase and buffer), into the microwell array (410). In some embodiments, nucleic acid constructs can be dispensed from one donor microwell array into an acceptor microwell array that aligns with the wells of the donor array. In other embodiments, genes are linked to beads and a pool of beads is added to the microwells. The pattern of the nucleic acid constructs in the microwell array can follow a Poisson distribution, thus, Poisson distribution can be used to determine the proper number of nucleic acid constructs in the loading solution, so that in most cases, at most one copy of each nucleic acid construct is added to one microwell. In some other embodiments, multiple nucleic acid constructs can be added to each microwell. The microwell array can be sealed by a movable plate (260), or sealed by a layer of oil, or enclosed in a 100% humidity chamber to prevent or minimize liquid evaporation.

A single copy of DNA can be amplified, e.g., using PCR or isothermal amplification methods (420). In vitro transcription/translation reagents and/or substrates are then added to the microwells (430). The in vitro transcription/translation reagents and substrates can be added before, at the same time, or after the beads are added to the microwells (430). The microwells can then be sealed, e.g., with oil or other hydrophobic liquid, or a physical cap structure (260), e.g., a glass, silicon rubber sheet, or some appropriate cover.

The nucleic acid constructs can include a sequence encoding an affinity tag, such as his-tag, FLAG-tag, or SNAP-tag. The polypeptides generated during IVTT (440) can be immobilized on protein-binding beads, e.g., through affinity binding. In some other embodiments, the surfaces of the microwells are functionalized using affinity tags or commonly used antibodies to tags such as FLAG, or SNAP, to enable immobilization of the synthesized polypeptides directly in the microwells.

Many cell-free protein synthesis reagents have been developed (see this review for comparison of common commercial cell-free systems (see, e.g., Chong, “Overview of cell-free protein synthesis: historic landmarks, commercial systems, and expanding applications,” Curr. Protoc. Mol. Biol., 2014 Oct. 1; 108:16.30.1-11. doi: 10.1002/0471142727.mb1630s108). In some embodiments, the system used in the present methods is a cell-extract based cell-free protein synthesis system, such as TnT® Quick Coupled Transcription/Translation System (Promega, Madison, Wis.) or other similar cell-extract based systems.

In some embodiments, the system used in the present methods is the PURExpress® system (New England Biolabs, Ipswich, Mass.) or other similar systems that are composed of recombinant or purified components and provide minimal contaminating background activities for direct downstream biological assays. In the PURExpress system, mRNA is translated into protein using aminoacyl tRNA intermediates and ribosomes consisting of dozens of proteins and three ribosomal RNAs in prokaryotes. To complete the translation of one open reading frame (ORF) encoded in the mRNA sequence, three reaction steps proceed on the ribosome: initiation, elongation, and termination. These reaction steps are followed by a ribosome recycling step to re-initiate translation. Several translation factors take part in each translation step: three initiation factors (IF1, IF2, and IF3), three elongation factors (EF-G, EFTu, and EF-Ts), three release (termination) factors (RF1, RF2, and RF3), and ribosome recycling factor (RRF). In addition, three other reactions are added to facilitate protein synthesis: transcription to synthesize mRNA, aminoacylation of tRNAs, and energy source regeneration. Thus, T7 RNA polymerase, pyrophosphatase, aminoacyl-tRNA synthetases, creatine kinase, myokinase, and nucleoside-diphosphate kinase are also incorporated into the system.

All factors in the PURExpress system are individually purified to remove contaminating activities such as nuclease and protease activities, and thus can significantly decrease the background signals for many downstream assays. DNA, RNA, and protein molecules are additionally more stable in such purified cell-free systems, which can increase the sensitivity in in vitro platforms that couple gene synthesis with protein synthesis and direct functional assays. Altogether, recombinant and synthetic cell-free IVTT systems such as PURExpress enable the same speed and high throughput of synthesis as cell-lysate based IVTT systems but allow greater experimenter control for reaction cofactors and decreased background for more sensitive readouts. A detailed description of this system is described, e.g., in Shimizu et al., “Protein synthesis by pure translation systems,” Methods, 36.3:299-304 (2005) and in U.S. Pat. No. 9,371,598, which are incorporated herein by reference in their entireties.

Following incubation of the reaction constructs, in vitro transcription/translation reagents, and substrates for a sufficient period of time, various methods can be used to detect and/or quantify the bioactivities of polypeptides, e.g., by fluorometric readout.

### Protein and Nucleic Acid Assays

Once the RNA and polypeptides are synthesized, the design of the microwell system described herein with the ability for fluid exchange enable a versatile set of reactions. While there are many possible reactions, the preferred embodiments are those that have a functional readout that is converted to a signal capable of high throughput readouts compatible with the scale of the microwell arrays.

In some aspects, these may utilize fluorometric readouts that provide a fluorescent signal that is proportional to the amount of desired reactants that formed. Substrates that are to be modified can be tagged with fluorescent/quencher pairs that in the unmodified state of the substrate do not fluoresce, but upon modification, the separation of the fluorescent dye from the quencher enables a readout of reaction progress. Additionally, the presence of IVTT solution enables synthesis of fluorescent proteins as a potential readout, as previously demonstrated (Cui, N. et al., “A mix-and-read drop-based in vitro two-hybrid method for screening high-affinity peptide binders,” Sci. Rep. 6, 22575; doi: 10.1038/srep22575 (2016)).

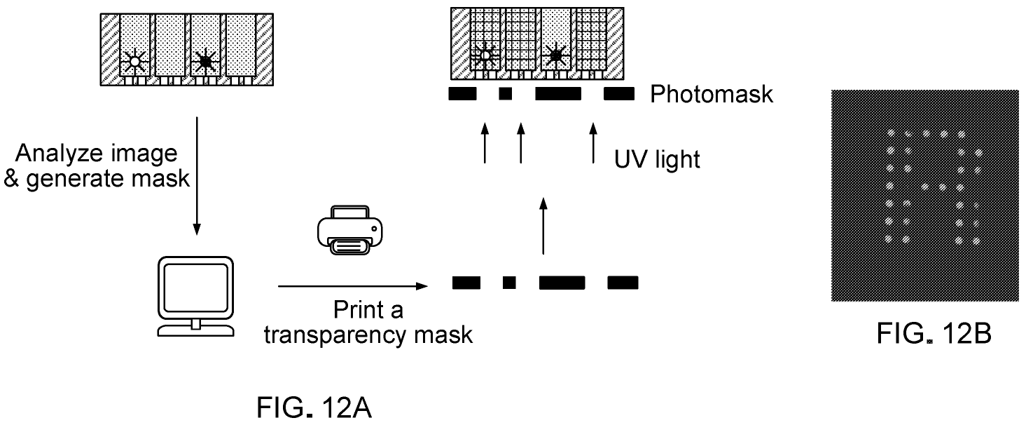

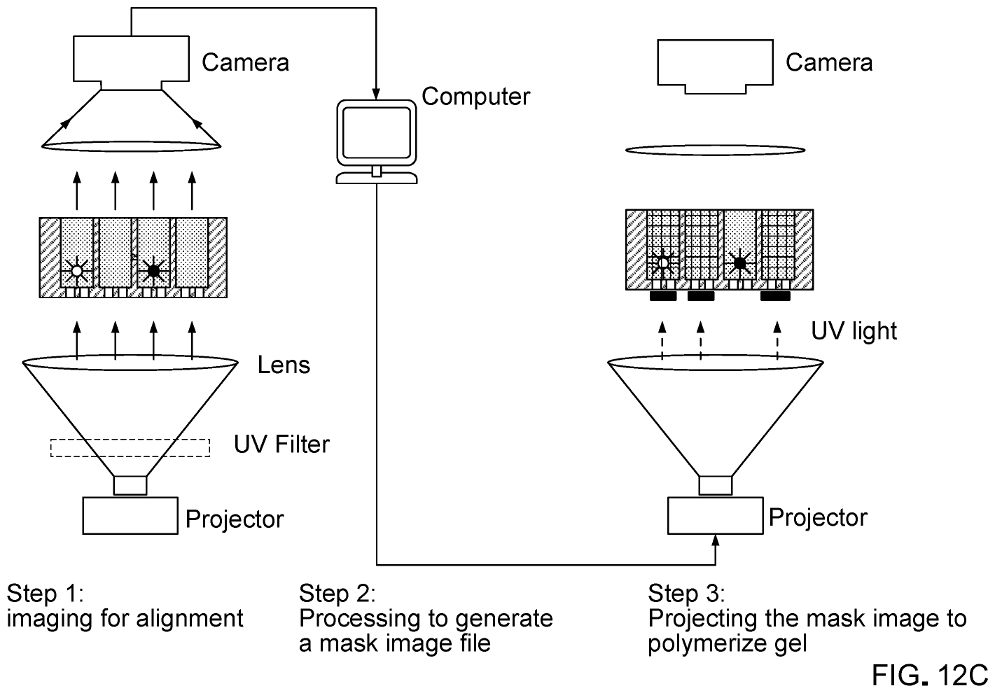

For detection of DNA modification, aspects of the aforementioned fluorescent assays can be adapted as follows: a DNA or RNA fragment can be labeled with a fluorophore and quencher, which upon cleavage will dissociate and generate a fluorescent signal. This enables the detection of RNA nuclease, ssDNA nuclease, DNA nicking, dsDNA nuclease, as well as insertion/deletion activities. For insertion/deletion activities, nucleic acid segments containing quencher or fluorophore elements are inserted into a targeted sequence, either enhancing or disrupting activity. Additionally, these fluorophore modifying readout probes may be delivered in cis or trans configuration with the original nucleic acid fragments encoding the construct(s). The cis targeting can be enabled by having the synthesized nucleic acid construct contain the fluorescent/quencher target as well. This potentially enables testing different substrates for each construct, or enables an all-in-one delivery a gene encoding a protein effector and its potential substrate on a single nucleic acid construct (FIG. 12). Trans targeting is another embodiment in which a fluorophore/quencher labeled DNA fragments is delivered as a common reagent across the microwells. In one aspect to direct substrates to different constructs, the substrates may be barcoded to enable targeting to specific beads or microwells, thus enabling greater specificity than would otherwise be enabled by delivering a common reagent in trans configuration.

The utilization of IVTT to synthesize a fluorescent protein in response to a DNA modification event provides additional possibilities to the readout of DNA modifications. In reactions classified as fluorescence restoration assays, a DNA/RNA encoding a fluorescent protein is either disrupted or restored using a DNA/RNA modifying effector, thus revealing a detectable change in protein activity. In one aspect, there are two constructs expressing different fluorescent proteins that can be spectrally distinguished; one channel acts as an internal control of nucleic acid loading and expression levels, while the other serves as the readout.

Beyond fluorescence, an alternative readout capable of scale compatible with ultrahigh throughput protein discovery is next generation sequencing. In one aspect, a construct encoding a nucleic acid target substrate and effector gene expressing the polypeptide/RNA of interest is immobilized in a microwell or to a microbead. Successful reconstitution of the effector system and nucleic acid modification results in release of the construct sequence encoding the modified target into the solution for collection and identification by next generation sequencing. The nucleic acid target substrate can either be located on the same gene synthesis product (cis), as suggested in FIG. 12, or on another fragment loaded separately (trans). These released nucleic acid strands can be eluted from the microwells with a gentle wash, allowing the cleaved fragments to be collected, concentrated, and sequenced to identify the target and effector responsible. This direct cleavage event enables the detection of dsDNA nuclease, DNA nicking, ssDNA cleavage activities, as well as insertion activities (in which a known cleavage site for a site-specific nuclease can be used as the insertion product, so that a successful insertion event results in a positive secondary cleavage event of the inserted product).

Example 1 below demonstrates the power of an all-in-one DNA cleavage readout assay that starts with a synthesized DNA product and proceeds to the DNA cleavage readout in a single reaction chamber. This reaction can be further miniaturized and the readout can be converted to released nucleic acids or a fluorometric readout for use in microwells.

In another aspect, all microwells and/or microbeads are loaded with nucleic acid barcode sequences that can be released in response to an external stimulus, such as light activated nuclease or other chemistries that enable a localized release of nucleic acids. In this embodiment, the reaction of interest is indirectly coupled to the nucleic acid release mechanism, such that positive readouts activate methods such as a scanning laser to selectively excite and release nucleic acids for identification.

These non-limiting embodiments serve to highlight the versatility and potential of the platform. Additional assays and readouts that are compatible with the ultrahigh throughput protein discovery platform are expected and can be modular with the other components described herein.

### Nucleic Acid Sequence Identification

Various methods are available to determine the sequences of the nucleic acid constructs on the blotting plate or nucleic acid construct array (540). All of these different methods can be computer-controlled and automated. Without wishing to be limited, there are three broad embodiments of methods and systems for selecting and identifying the nucleic acid construct; the first utilizes a pre-decoded or registered construct arrays, in which prior to the reaction the exact spatial location of each construct is known so that a subsequent positive signal can immediately be associated with the sequence that gave rise to it. The second is identifying and collecting the constructs of interest directly in the microwell plate, whether that is using technologies such as robotic picking of the loaded beads or eluting cleaved fragments of nucleic acids. The third takes specific advantage of the reagent exchange capabilities of the advanced microwells described herein to elute a separate duplicate “blotting plate” whose identities can be read out separate of the actual reaction.

In the first embodiment utilizing pre-decoded arrays, numerous aspects exist. One instance is a microwell array that contains oligonucleotide sequences directly synthesized onto the functionalized surface of the individual microwells. Fluid jetting systems can be utilized to specifically synthesize oligonucleotides in specific wells so that the sequence identities of each well are well-defined. Thus, when a gene pool containing complementary barcode sequences are flowed over the array to load the microwells, specific full-length constructs will then hybridize to their pre-defined locations. In another instance, a randomly loaded array such as a microwell or bead array is first decoded utilizing methods, such as those described in U.S. Pat. No. 6,620,584B1, prior to hybridization with full-length genes of interest and performing the subsequent protein synthesis and functional assays.

In the second embodiment, the reaction array can be directly manipulated to yield the identity of the signals of interest. In one aspect, this can be performed by mechanical methods such as miniaturized robotics or piezoelectric actuators (Alogla et al. “Micro-tweezers: Design, fabrication, simulation and testing of a pneumatically actuated micro-gripper for micromanipulation and microtactile sensing.” Sensors and Actuators A: Physical 236 (2015): 394-404) to select the beads from wells of interest. In other embodiments, optical or laser based selection methods (Chen et al., “High-throughput analysis and protein engineering using microcapillary arrays,” Nature Chem. Biol., 12.2 (2016): 76) are able to select and transfer samples of interest for collection and downstream analysis.

Other methods of direct manipulation include the direct release of DNA fragments or barcodes for sequencing, as described in the “Protein and Nucleic Acid Assays” section.

In some embodiments, the constructs in the wells may be able to be directly assayed and then sequenced on a flow cell. Without wishing to be limited, in the instance in which sequencing by synthesis is performed as the readout, the microwells utilized as well as the nucleotide acid sequence constructs used have the necessary adaptor sequences on the microwell surface and at the gene terminals, respectively. After functional assays, the nucleic acid constructs can be directly sequenced on the patterned microwell array to reveal their identity.

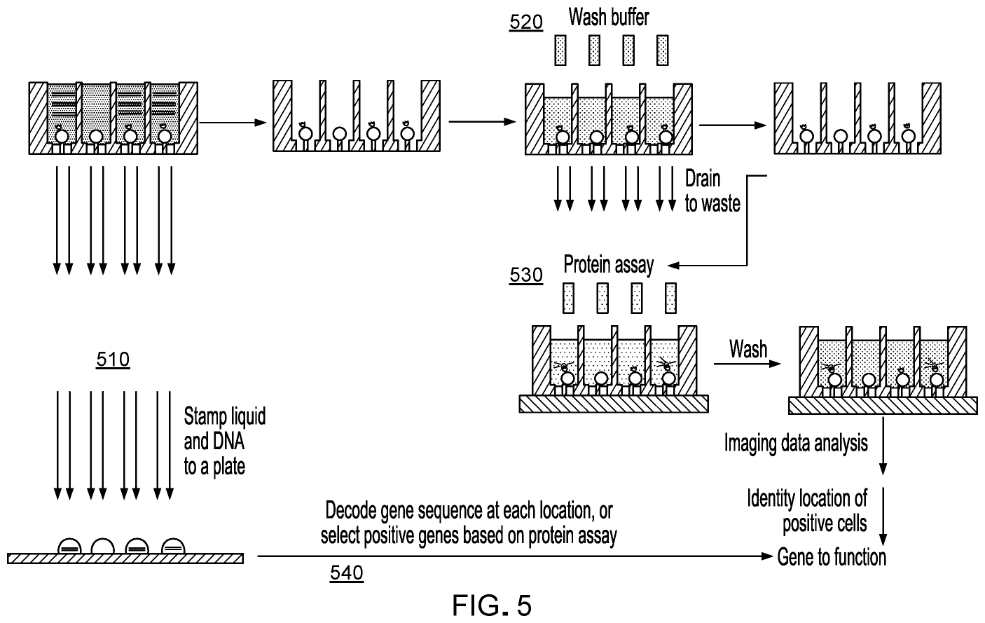

In the third embodiment, the nucleic acid contents of the microwells are partially transferred to a ‘blotting plate’ that carries the same spatial localization of nucleic acids as the main reaction microwell array, but enables greater flexibility in reading out the identity of individual locations. FIG. 5 shows a schematic of an assay that can be used to identify polypeptides of interest, and the nucleic acid constructs used to encode them. As shown in FIG. 5, nucleic acid constructs and polypeptides in the microwells are separated by stamping or centrifugation to form a nucleic acid construct array (510), wherein each location in the nucleic acid construct array has a specific coordinate that corresponds to the specific microwell in the array. In some embodiments, one or more liquids in the microwells can be transferred to a blotting plate. The blotting plate can have hydrophobic barriers with a pattern that is similar to the microwell array. The hydrophobic barriers can be generated using various methods known in the art, e.g., a wax printer, printing, or photolithography. Hydrophobic barrier on the plate can prevent liquid cross-contamination (e.g., cross-contaminating with liquids in adjacent microwells). In other embodiments, biophysical processes such as applying an electric field, can enable migration of the nucleic acids from the microwell into the blotting plate.

In some embodiments, the nucleic acid construct (e.g., DNA) arrays can be decoded by sequentially hybridizing different fluorescence probes to the nucleic acid construct. Methods of decoding nucleic acid constructs using fluorescence probes are described in detail, e.g., in U.S. Pat. No. 6,620,584, which is incorporated herein by reference in its entirety.

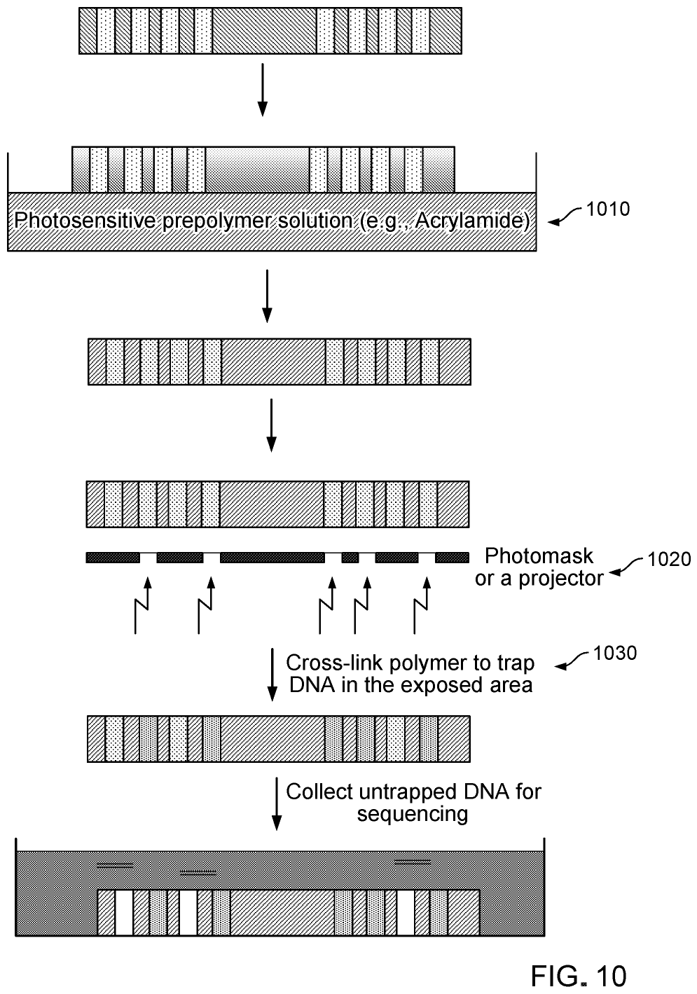

In some embodiments, light can be used to retain or release specific nucleic acid constructs (e.g., DNA) at selected locations. As shown in FIG. 10, a photosensitive monomer solution (e.g., acrylamide) can be added to a blotting plate (1010). A photomask is then applied to the blotting plate (1020). The blotting plate is then exposed to a light source (1030) at selected locations, e.g., that correspond to the coordinates of selected, marked microwells. Monomers at areas exposed to light are cross-linked and trap nucleic acid constructs at these locations. With respect to unexposed region, nucleic acid constructs are rinsed out, purified, and analyzed, e.g., by sequencing. In some other embodiments, nucleic acid constructs that are trapped by cross-linked polymer are released and sequenced. In some embodiments, a projector or digital micromirror device (DMD) with a predetermined light pattern can be used to cross-link polymers at selected locations.

In some other embodiments, all nucleic acid constructs (e.g., DNA) are trapped by a polymer, and light is used to break polymers and thus selectively release nucleic acid constructs at specific locations. In some embodiments, nucleic acid constructs (e.g., DNA) are captured on the blotting plate by light-sensitive binding, and light can be used to disrupt the binding and selectively release the nucleic acid constructs.

In some embodiments, a fluid jetting system (e.g., inkjets) can selectively add hydrophobic materials (e.g., wax) to seal the nucleic acid constructs at appropriate locations. The nucleic acid constructs at other locations (unsealed by hydrophobic material) can be rinsed out and analyzed.

In some embodiments, laser microdissection can be used to cut out areas in the blotting plate and nucleic acid constructs at the cut-out locations or at the remaining locations can be selectively released and sequenced. In some other embodiments, a robotic arm is used to collect nucleic acid constructs mechanically on the blotting plate.

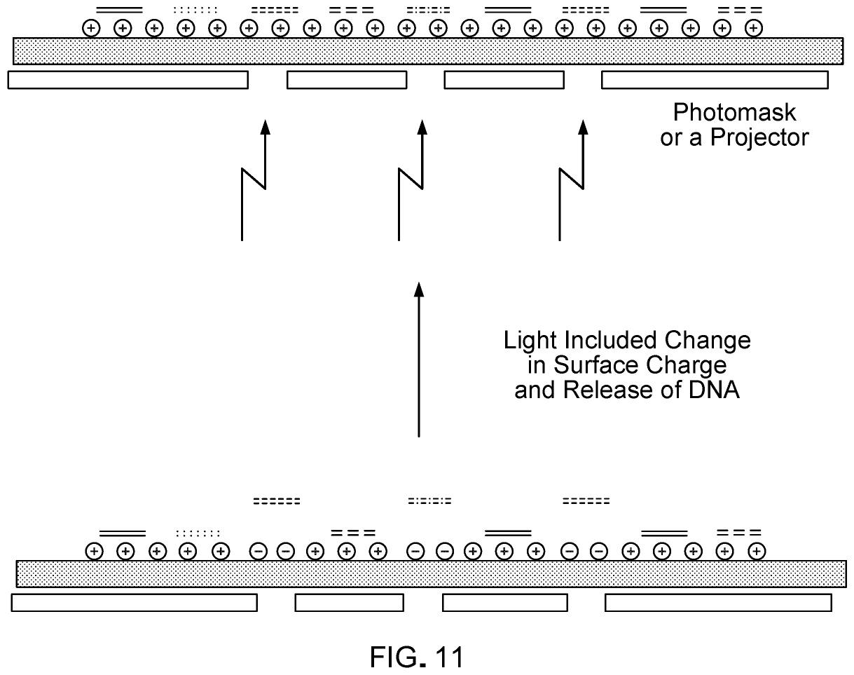

As shown in FIG. 11, a surface with positive charge can be used to capture nucleic acid constructs (e.g., DNA), which usually have negative charges. The surface can be made by coating a layer or a self-assembled monolayer of photosensitive material on glass, silicon, gold, plastic, or similar materials. The material can be coated through casting, spin-coating, chemical vapor deposition, layer-by-layer deposition, and similar methods. The coating material can be photosensitive polymers or photochromic molecules, which transit between chemical structures upon absorption of light. For example, spiropyran containing polymers changes to its isomer merocyanine after light irradiation, and causing a change in net charge on the surface (Gumbley et al., “Reversible Photochemical Tuning of Net Charge Separation from Contact Electrification,” ACS Applied Materials & Interfaces 6.11 (2014): 8754-8761).

When the selected areas are exposed to light, the light causes the surface charge to switch from positive to negative, e.g., by switching between two different chemical structures, by breaking chemical bonds, or by inducing a pH change. Nucleic acid constructs, which are typically negatively charged, at the exposed area are then repelled and released from the surface and can be further manipulated, analyzed, or sequenced. In some embodiments, the unreleased nucleic acid constructs are also sequenced.

In some embodiments, nucleic acid constructs that encode polypeptides with desired properties are released and sequenced (“positive selection”). In some other embodiments, nucleic acid constructs that encode polypeptides without desired properties are released and washed away first, and the remaining nucleic acid constructs on the blotting plate are then released (e.g., by a stronger wash buffer) and sequenced (“negative selection”).

In some embodiments, protein to be screened can cleave bonds or trigger an action of releasing, e.g., nuclease. The positive hits of these proteins could automatically release polynucleotides used to make these proteins. After IVTT and incubation, all the liquid in the microwells can be pooled together to purify, sequence or analyze polynucleotides in the liquid. The nucleotides discovered are positive hits.

In some embodiments, target contents (e.g., beads, proteins, or nucleic acid constructs) can be selectively collected by inducing polymerization of a polymer solution in microcells. As shown in FIG. 12A, the signals from the microwells can be analyzed (e.g., fluorometric readouts). Based on the signals, a photomask can be generated. In some embodiments, the photomask is generated by printing ink on a transparent material. Then the photomask can be applied to the microwells. The microwells can be exposed to light (e.g., UV light), and the light can induce polymerization of the solution in the microwells. The photomask can have a pattern based on the microwells of interest.

In some embodiments, the contents of no interest are trapped in the microwells by the polymer. Then the target contents of interest can be collected by methods described herein.

In some embodiments, the contents that are of no interest can be washed away first, while the target contents of interest are retained in the microwells due to polymerization of the polymer solution. Thereafter, the target contents of interest can be collected from the microwells.

FIG. 12B is a fluorescence image of a microwell array showing that GFP proteins in certain microwells were successfully trapped by photo-polymerized polymer in those microwells. GFP in other wells was washed away.

FIG. 12C is a schematic overview of a fully automated system that images microwells, analyzes images by a computer, generates a specific mask through a projector, and traps target contents through photo-polymerization. As shown in FIG. 12C, the array can be aligned with a detector for imaging. The signals are analyzed and can be used to determine microwells of interest. Based on the analysis, a photomask can be generated. Alternatively, a projector can be used to generate an image with a specific pattern so that selected microwells are exposed to light (e.g., UV light). The light can induce polymerization in the selected microwells (e.g., microwells of interest) without the need for a photomask.

### Functionalizing Microwell Surfaces

The disclosure also provides various methods to functionalize microwell surfaces, and can be adapted for a wide range of uses.

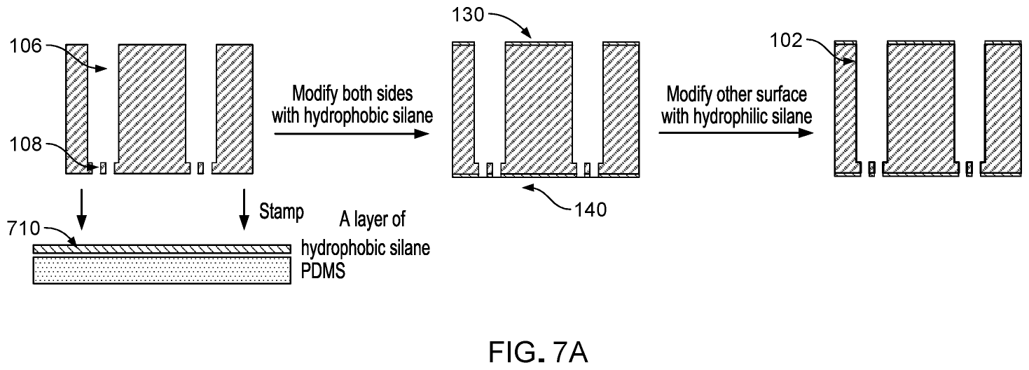

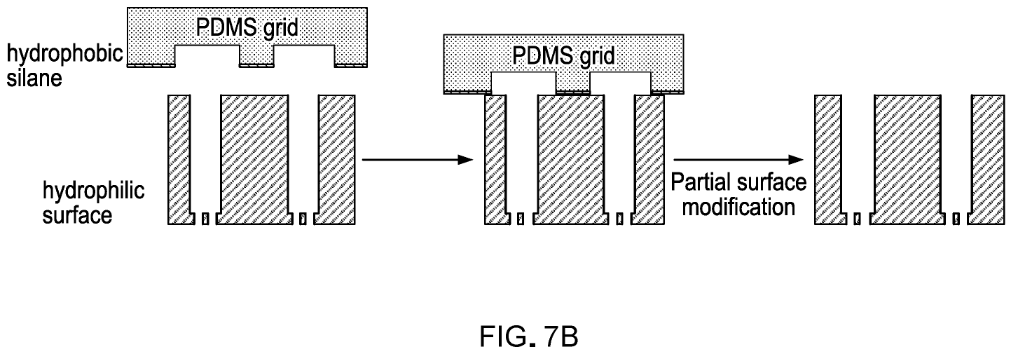

In some embodiments, a layer of hydrophobic material, (e.g., silane, thiol) can be spin-coated on a flat surface, such as PDMS, silicon, glass, or gold (FIG. 7A). The hydrophobic material (e.g., n-octadecyltrichlorosilane, (heptadecafluoro-1,1,2,2-tetrahydrodecyl) trimethoxysilane, perfluorodecanethiol) can form a thin layer (710) or a self-assembled monolayer on the surface. When microwell arrays contact the hydrophobic material, the hydrophobic material will modify only the surface it contacts. The top surface (130) and the bottom surface (140) of the microwell array can both be modified by hydrophobic material). Then the chip can be flooded with hydrophilic material (e.g., (3-Aminopropyl)triethoxysilane), thus modifying the inner sidewalls (102) of the microwells. In this process, the area that has been modified with hydrophobic material will not be further modified by the hydrophilic material.

In some embodiments, the microwell surface or part thereof can be functionalized using a microstructured surface, e.g., polydimethylsiloxane (PDMS) (FIG. 7B). The microstructured surface can print or stamp certain area of the microwell surface, e.g., with hydrophobic material, e.g., silane. In some embodiments, the microwell surface is modified with a specific pattern.

In some embodiments, the chip can be flooded with hydrophilic material first to cover surface, and then use method (e.g., polishing) to selectively remove coating at the outside surface. In some embodiments, proteins (e.g., receptors, ligands, or antibodies) can be attached to surfaces inside the microwells. In some embodiments, oligo-conjugated antibodies can be used to functionalize flow-cell surfaces or oligos bound to microwell surfaces. In some embodiments, the protein binding can be used to sequester proteins in the microwells. In some embodiments, the protein binding can be detected, e.g., by Surface Plasmon Resonance (SPR).

Numerous methods of attaching proteins to microwell surfaces are known in the art. Oligonucleotide-protein conjugates can be used in numerous applications for diagnostic and therapeutic purposes. Proteins (e.g., antibody molecules) can include a number of functional groups suitable for modification or conjugation purposes. In some embodiments, oligonucleotide-protein constructs can be cross-linked through lysine ϵ-amine and N-terminal α-amine groups. In some embodiments, the protein is hydrazine-activated through a reaction between the amine group and the Sulfo-S-HyNic crosslinker. The S-HyNic (succinimidyl-6-hydrazino-nicotinamide) hetero-bifunctional crosslinker is used in Chromalink™ technology. Sulfo-S-HyNic is a water soluble analog of S-HyNic. The S-HyNic analog reacts with primary amines on proteins (amino group of lysine) or amino-modified oligonucleotides or surfaces, introducing a HyNic (6-hydrazino-nicotinamide) linker that forms stable covalent conjugates with biomolecules possessing 4FB (4-formylbenzamide) incorporated linkers. Sulfo-S-HyNic can also be used for incorporating HyNic linkers on amino-surfaces or biomolecules. The hydrazine-activated protein (e.g., antibody) is then linked to an aldehyde-activated oligonucleotide.

In some embodiments, the amine group in the protein can react with a maleimide-activated biopolymer, thus linking the protein with the biopolymer (e.g., oligonucleotides, polypeptides, and polysaccharides). In some embodiments, carboxylate groups can also be used to couple with another molecule using the C-terminal end, or with aspartic acid and/or glutamic acid residues.

In some embodiments, the protein is an antibody. Amine and carboxylate groups are as plentiful in antibodies as they are in most proteins, and the distribution of these functional groups is nearly uniform on the surface of antibodies. Thus, if some of the modified or conjugated residues are located on the antigen binding sites, the methods may produce oligonucleotide-antibody conjugates that are only partially active or inactive and thus may not bind to the antigen. In such cases, an alternative conjugation method can be used that involves a thiol reactive group by selectively cleaving an antibody with a reducing agent to create two half-antibody molecules, or using smaller antibody fragments such as Fab′ fragments.

In some embodiments, conjugation done using hinge area-SH groups can orient the attached oligonucleotide away from the antigen binding regions, thus preventing blockage of these sites and preserving activity. Reduction in a hinge region by a reducing agent, e.g., tris(2-carboxyethyl)phosphine (TCEP), dithiothreitol (DTT) or mercaptoethylamine (MEA), yields two half antibody molecules containing sulfhydryls. The sulfhydryl group can react with maleimide-activated biopolymers, forming an antibody-oligo conjugate through a thioether bond.

Other alternative methods of site-directed conjugation of antibody molecules can take place at carbohydrate chains, e.g., at the CH₂ domain within the Fc region. Upon periodate oxidation an aldehyde group can be introduced to the antibody, which can react with an amine-modified oligonucleotide.

In other embodiments, the biopolymers can bind to the surfaces of microwells through complementary binding between oligonucleotides, thus attaching the proteins to the microwells.

Furthermore, products from the nucleic acid constructs can be immobilized on inner surfaces of microwells by nucleic acid conjugated antibodies that specifically bind to the gene products.

### Synthesis and Sequencing of Pooled DNA Libraries for High Throughput Biodiscovery

Nucleic acid constructs (DNA or RNA constructs) can be synthesized based on the nucleotide sequences from metagenome mining or engineered non-naturally occurring sequences. As used herein, a nucleic acid construct is an artificially constructed segment of nucleic acid molecule that can be transcribed and/or translated into a peptide, polypeptide, or protein. The nucleic acid constructs described herein can include a promoter sequence, followed by a desired coding sequence, and a transcription termination or polyadenylation signal sequence. The nucleic acid constructs can either be obtained pre-synthesized as full-length constructs in either pooled or arrayed forms, or can be directly synthesized in the system.

The present disclosure provides a method for the synthesis, sequencing, and optionally, the isolation of individual target DNA construct molecules resulting from the synthesis of one or more target DNA constructs. The present disclosure also provides a method for the sequencing, and optionally, the isolation of individual target DNA constructs from a homogeneous or heterogeneous population of circular or linear DNA molecules.

In one aspect, the disclosure provides a method for the assembly of one or more target DNA sequences, such that individual target DNA molecules can be fully or partially sequence verified, and isolated. The method includes or consists of multiple seed oligonucleotides or DNA fragments composing one or more target DNA constructs; a target subgroup barcode specifying subgroups of one or more target DNA constructs; and a unique molecular identifier specifying individual target DNA molecules resulting from assembly methods, such as polymerase chain assembly (PCA), Gibson Assembly® (Synthetic Genomics Inc.), or Golden Gate Assembly (tUMI). Polymerase chain assembly can be used to assemble complete oligonucleotides with complimentary overlapping regions. Alternatively, Gibson Assembly or Golden Gate Assembly can be used to assemble multiple double stranded DNA fragments with either overlapping ends or complimentary type-IIS restriction sites mediating the assembly of the target DNA construct.

In another aspect, the disclosure provides a method for sequencing of one or more subregions of interest within a population of homogeneous or heterogeneous DNA molecules. The method includes or consists of multiple DNA molecules composing one or more target DNA sequences of interest; a target subgroup barcode specifying subgroups of one or more target DNA constructs; and a unique molecular identifier specifying individual target DNA molecules resulting from PCA (tUMI).

### DNA Assembly in Pooled or Isolated Reactions

In some embodiments, the seed oligonucleotides composing one or more target DNA constructs are assembled in a single pooled PCA reaction.

In some embodiments, the PCA seed oligonucleotides contain target subgroup barcodes for hybridization or binding to beads or surface-immobilized oligonucleotides.

In some embodiments, surface immobilized oligonucleotides are contained within compartments, such as microwells or interspersed hydrophilic regions separated by hydrophobic regions. In such cases, each bead or surface compartment contains one or multiple oligonucleotides complimentary to one or multiple target subgroup barcodes or a binding moiety that specifically binds and sequesters different target subgroup barcodes.

In some embodiments, individual beads containing bound seed oligonucleotides corresponding to one or multiple target subgroups are encapsulated in individual emulsion droplets or microwells. In some embodiments, individual surface compartments containing bound seed oligonucleotides corresponding to one or multiple target subgroups are encapsulated as sequestered reaction chambers based on physical or chemical properties of the surface (ex: microwell, interspersed hydrophilic regions, or localized regions within a single reaction chamber).

In some embodiments, once encapsulated in an emulsion droplet or sequestered reaction chambers, the seed oligonucleotides are released from the bead or surface. In some embodiments, the target subgroup barcodes are cleaved from the seed oligonucleotides prior to PCA. In some embodiments, the pooled PCA reaction or sequestered PCA reactions also contain terminal primers for amplification of fully assembled target DNA sequences. For example, application WO2012154201 describes the synthesis of multiple target DNA constructs in a single pooled reaction, and is incorporated herein by reference in its entirety. Application US20150051117 describes the sequestration of seed oligonucleotides for a single target DNA construct on beads, encapsulation of individual beads in emulsion droplets, and performance of PCA in droplets, and is incorporated herein by reference in its entirety.

DNA Synthesis within the Microwell Arrays

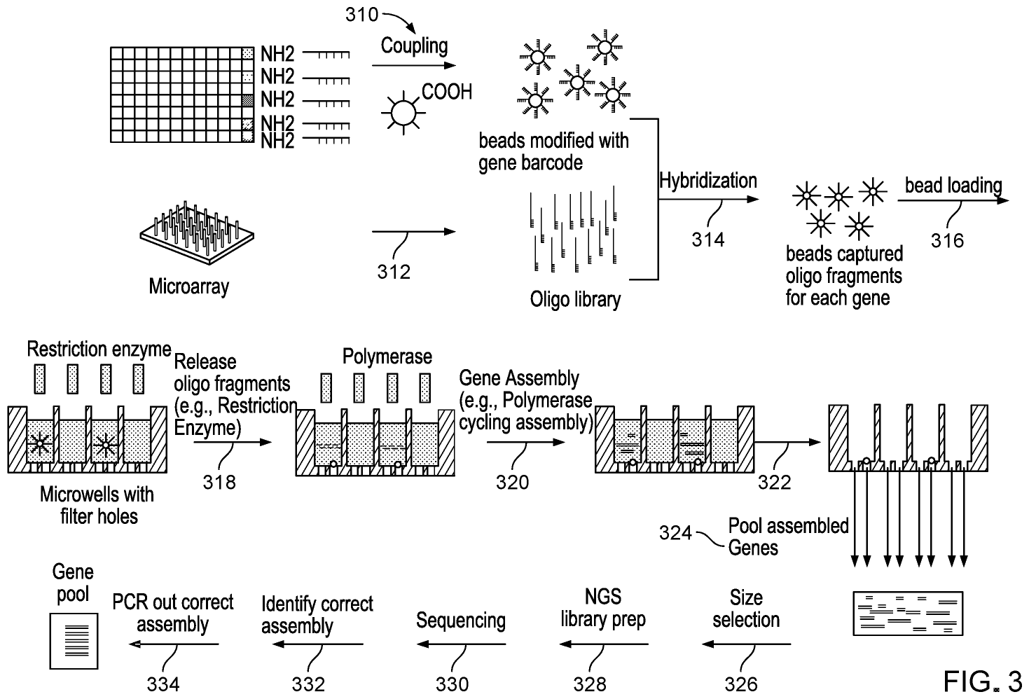

FIG. 3 shows one example of synthesizing nucleic acid constructs. Single stranded barcode oligonucleotides are synthesized. Carboxylic acid modified beads can then be coupled with these individually synthesized single stranded barcode oligonucleotides with amine group through a standard coupling chemistry (310). One or more sets of barcoded double stranded oligonucleotide subsequences are also synthesized (312). One set of barcoded, double-stranded oligonucleotide subsequences defines an oligonucleotide set corresponding to a particular nucleic acid sequence of interest. Each barcoded, double-stranded oligonucleotide subsequences in one set has a common single-stranded barcode oligonucleotide, and can attach to a bead having a complementary common single-stranded barcode by hybridization (314). Other sets of barcoded, double-stranded oligonucleotide subsequences can also be synthesized; and these subsequences together provide the full length of other nucleic acid sequences of interest. These subsequences can also attach to beads having a different complementary common, single-stranded barcode. The assembly methods are described in detail, e.g., in US20150051117A1, which is incorporated herein by reference in its entirety.

These beads are then loaded, manually or automatically, into the microwell array (316). The concentration of the beads is sufficiently low so that at most one bead is loaded into one microwell, e.g., each microwell then contains zero or one bead. Double-stranded oligonucleotides (e.g., DNA segments) are released into the microwell by restriction enzyme digestion (318). These oligonucleotides can together form a full length of nucleic acid sequence of interest (e.g., gene sequence), and are linked together because of pre-designed overhanging sequences. These oligonucleotides are then automatically assembled by polymerase cycling assembly (PCA). The one or more reaction solutions in the microwells are then emptied (322), and the nucleic acid constructs in the solution are pooled together (324). Nucleic acid constructs with appropriate lengths are selected, e.g., by gel electrophoresis (326).

The constructs with appropriate lengths are then used to prepare a next generation sequencing (NGS) library (328), and are automatically sequenced (330). Sequencing results are then analyzed to identify correct gene assembly (332). PCR primers can be designed to select and amplify only the correct gene assembly (334). The PCR products can then be used individually or pooled together for further use, e.g., screening sequences encoding proteins with desired properties.

### Labeling of Target DNA Molecules During PCA

In some embodiments, either or both of the tUMI and the target subgroup barcode are attached to terminal primers complimentary to terminal target DNA sequences that are used for amplification of assembled fragments during PCA, In some embodiments, terminal primers containing either one or both of the tUMI and target subgroup barcodes also contain a primer binding sequence 5′ of the tUMI or target subgroup barcode on one or both of the terminal primers. In some embodiments, primers for one or more primer binding sequences 5′ of the tUMI or target subgroup barcode are added to the PCA reaction. In some embodiments, primers for one or more primer binding sequences 5′ of the tUMI or target subgroup barcode may be added at 10¹, 10², 10³, 10⁴, 10⁵, or 10⁶ molar excess to PCA terminal primers containing the tUMI Importantly, this approach can be used to create a sparse set of tUMI labeled products that are further amplified by a primer 5′ to the tUMI.
Labeling of Target DNA Molecules from Circular or Linear DNA Populations

In some embodiments, either or both of the tUMI and the target subgroup barcode are attached to terminal primers complimentary to sequences flanking a target. DNA region of interest within a homogeneous or heterogeneous population of circular or linear DNA molecules. In some embodiments, terminal primers containing either one or both of the JAI′ and target subgroup barcodes also contain a primer binding sequence 5′ of the tUMI or target subgroup barcode on one or both of the terminal primers. In some embodiments, primers for one or more primer binding sequences 5′ of the tUMI or target subgroup barcode are added to the PCA reaction. In some embodiments, primers for primers for one or more primer binding sequences 5′ of the tUMI or target subgroup barcode may be added at 10¹, 10² 10³, 10⁴, 10⁵, or 10⁶ molar excess of PCA terminal primers containing the tUMI, importantly, this approach can be used to create a sparse set of tUMI labeled products that are further amplified by a primer 5′ to the tUMI.

### Amplification of Target DNA Subregions

In some embodiments, one or more target DNA constructs resulting from a pooled PCA reaction or multiple sequestered PCA reactions will each contain a target unique molecular identifier and optionally a target subgroup barcode. In some embodiments, one or more regions of interest amplified from circular or linear DNA will each contain a target unique molecular identifier and optionally a target subgroup barcode. In some embodiments, each DNA fragment molecule labeled with a target UMI (tUMI) has been amplified.

In some embodiments, multiple subregions of each tUMI-labeled DNA fragment amplified using a primer that binds a site 5′ of the tUMI and multiple different primers amplifying from the opposing side of the DNA fragment, such that each subregion contains the tUMI-containing end of the fragments and stepwise truncations from the opposite side of the fragment.

Association of Target DNA UMI with Target DNA Subregions

In some embodiments, the 5′ most regions of the primer pairs used to amplify target DNA subregions contain complimentary restriction sites. The resulting complimentary restriction sites on either side of the amplified subregions can be used to circularize the subregions using restriction and ligation. In some embodiments, restriction sites are not included, and the blunt ends of amplified subregions are ligated to create a circular product.

### Preparation of Target DNA Subregion Sequencing Library

Circularized subregion products contain a tUMI immediately proximal to subregions tiling the target DNA construct. In some embodiments, a primer 5′ to the tUMI, and another primer approximately up to 100 nt, 200 nt, 300 nt, 400 nt, or 500 nt 3′ to the tUMI are used to amplify short sections of the target DNA subregions attached to the tUMI. In some embodiments, these primers contain additional handle sequences for next generation sequencing (FIG. 3, steps 328 and 330).

### Reconstruction of Complete Target DNA Sequences

Next generation sequencing provides, at a minimum, the sequence of a tUMI specifying a particular target DNA construct molecule and the sequences of one or more subregions tiling the fragment. Grouping of subregions by tUMI sequence can provide the complete reconstruction of the target DNA construct (FIG. 3, step 332).

Nanopore Sequencing of tUMI-Labeled Target DNAs

Nanopore sequencing provides long reads capable of sequencing long target DNA constructs labeled with tUMIs in a single read. Although the error rate of sequencing-by-synthesis based next generation sequencing is much lower than Nanopore, amplification of target DNA construct molecules labeled with individual tUMIs could provide multiple coverage of a single tUMI labeled molecule by nanopore sequencing, enabling reconstruction of a consensus sequence for each labeled molecule (FIG. 3, steps 330 and 332). In some embodiments, tUMI labeled target DNA constructs are subjected to nanopore sequencing without amplification.

### Isolation of Specific Target DNA Molecules

In some embodiments, the tUMI sequence and optionally flanking sequence can serve as a probe or primer binding sequence that can be used to uniquely isolate a specific tUMI-labeled target DNA construct. In some embodiments, multiplexing of such primers or probes can be used to select for isolation of one or more target DNA constructs in a single reaction. In some embodiments, one or more probes specific to one or more target DNA constructs are used to affinity purify the fragments. In some embodiments, one or more primer pairs specific to one or more target DNA constructs are used to amplify and enrich for the fragments (334).

### EXAMPLES

The invention is further described in the following examples, which do not limit the scope of the invention described in the claims.

### Example 1: Coupled Expression and Assay from 1-Piece DNA

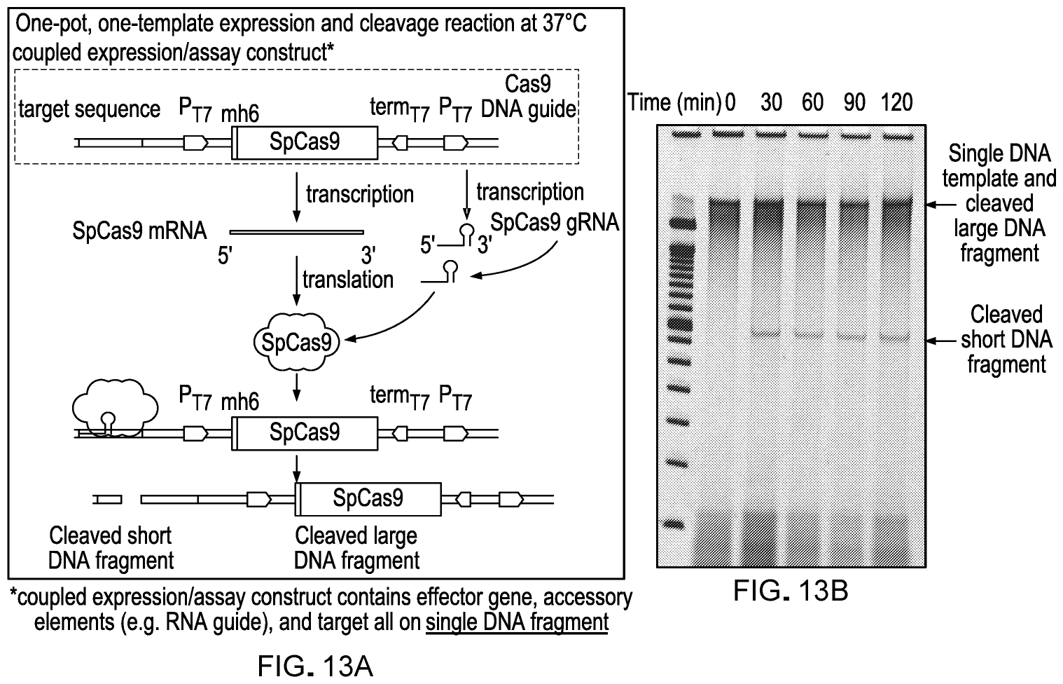

This example demonstrates an all-in-one DNA cleavage readout assay utilizing IVTT that starts with a synthesized DNA product and proceeds to the DNA cleavage readout rapidly and in a single reaction chamber. In this example, the protein of interest is a CRISPR-Cas9 nuclease from S. pyogenes (SpCas9). The DNA fragment for the assay contains, from 5′ to 3′, the target sequence, a T7 promoter, a bacterial-codon optimized SpCas9 effector protein with a mH6 tag at the N′ terminus, a T7 terminator, and then a second T7 promoter to express the noncoding single guide RNA for SpCas9 (FIG. 13A). The dsDNA is mixed with the IVTT mixture and incubated at 37 C for 0-120 minutes, with samples taken at 30 minute increments.

The nuclease reaction is directly read out from the reaction well by gel electrophoresis, displaying a short DNA fragment that is newly formed as the result of the cleavage activity of the SpCas9-sgRNA effector complex on the template DNA strand for IVTT (FIG. 13B). The result is apparent at 30 minutes into the reaction, suggesting that this can be a rapid readout for nuclease activity.

This demonstrates the full versatility of the proprietary IVTT reagent; we are able to observe all three macromolecule elements of the Central Dogma; protein and noncoding RNA are expressed and then complex together into a functional nuclease complex, enabling cleavage of the original DNA target. This reaction can be further miniaturized and the readout can be converted to released nucleic acids or a fluorometric readout for use in microwells.

### Example 2: Expressing Proteins in Microwells

The PURExpress® system was used to express green fluorescence protein (GFP) in microwells. Nucleic acid encoding GFP was coupled to magnetic beads (9 um diameter) through streptavidin-biotin linkage. The magnetic beads also contain red fluorescence dye for easy observation. An array of microwells (100 um well diameter, 300 um spacing) were fabricated using silicon wafer by a method shown in FIG. 6G Each microwell contains a plurality of filter holes (5 um diameter) made of silicon dioxide. The top and bottom surfaces of microwells were modified using n-Octadecyltrichlorosilane (FIG. 7B). The chip of microwells was dipped in acetone, ethanol, water, and PBS buffer subsequently to prime the wells with liquid. A suspension of beads containing GFP gene were added to the well-side of the chip, and the filter-side of the chip was placed on a wet KIMWIPES® to wick the liquid through the microwells. Beads were captured by the filter holes, and randomly distributed on the array. PURExpress solution was then loaded into microwells. The chip was then placed in a bath of mineral oil. In the oil bath, both sides of the microwells were wiped using a lint-free swab to remove excess amount of PURExpress® solution and to seal the microwells. The oil bath was heated to 37° C. and observed from the filter-side using an epifluorescence microscope for four hours.

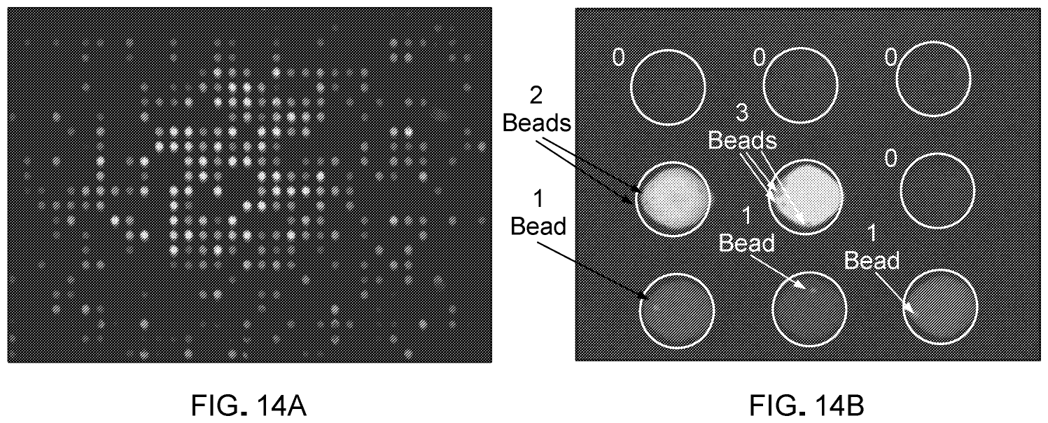

FIG. 14A is a fluorescence image showing that GFP were expressed inside the selected microwells in the array through in vitro transcription and translation (IVTT). FIG. 14B is an enlarged image of FIG. 14A and shows that the number of beads was correlated with the intensity of the GFP signals. A microwell that had 2 or 3 beads had stronger GFP signals as compared to a microwell with only one bead.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A microwell array system comprising
a microwell array comprising a plurality of isolated microwells, each microwell having side walls, a bottom wall, and a top opening, wherein the microwells are positioned in an array, and wherein each well comprises one or more filter holes arranged in the bottom wall of the microwell;
a cover, arranged to optionally and selectively cap one or more of the filter holes;
a reservoir to receive waste liquids exiting the microwells through the filter holes, through the top opening of microwells, or both;
a substrate to receive contents of one or more of the microwells deposited at one or more locations of a microarray (also known as a ‘blotting plate’), wherein each location has a known coordinate within the microarray;
a system for adding liquids to each microwell;
a system for adding microbeads to each microwell; and
a system for selecting and marking selected contents at specified locations in the microarray, and optionally, a system to decode contents with given coordinates.

2. The microwell array system of claim 1, wherein (i) the volume of each isolated microwell is about 0.5 picoliters to about 100 nanoliters (nl) or is less than 100 nanoliters (nl), 50 nl, 10 nl, 5 nl, 1 nl, 500 picoliters (pl), 250 pl, 100 pl, 50 pl, 25 pl, 20 pl, 15 pl, 10 pl, 5 pl, or 1 pl, (ii) each microwell has a diameter of from about 5 to 200 microns, or a diameter that is less than 200, 150, 100, 90, 80, 70, 60, 50, 40, 30, 20, or 10 microns, or (iii) each filter hole has a diameter of from about 0.5 to 150.0 microns.

3-4. (canceled)

5. The microwell array system of claim 1, wherein the microwell array has at least 5K, 10K, 50K, 100 K, 250 K, 500 K, 1 M, 5 M, 10 M, or 15 M microwells or the microwell array has at least 100, 1 K, 5 K, 10 K, 50K, or 100 K microwells per cm².

6. (canceled)

7. The microwell array system of claim 1, wherein the inner walls of each microwell are hydrophilic, and surfaces of the microwell array and of the cover are hydrophobic.

8. The microwell array system of claim 1, wherein the system for adding liquids: (i) adds liquids to each microwell via capillary force, (ii) comprises one or more microfluidic channels, (iii) comprises a liquid jetting system, (iv) comprises a pressure or vacuum pump, or (v) comprises a motor arranged to rotate the microwell array to distribute a liquid across a surface of the microwell array and into each microwell by spin-coating, optionally wherein the motor is controlled to spin sufficiently fast to remove excess liquids once the microwells are filled by the liquids.

9-13. (canceled)

14. The microwell array system of claim 1, wherein the diameter of the filter holes is smaller than a diameter of beads used with the system, or each microwell comprises two or more filter holes, wherein all filter holes are smaller than a diameter of beads used with the system and wherein second and any subsequent filter holes are smaller than the first filter hole.

15. (canceled)

16. The microwell array system of claim 1, wherein (i) the protein screening system further comprises a centrifugation system arranged to empty waste liquids in the microwells by centrifugation, (ii) the liquid in each microwell is deposited on the substrate by centrifugation or air pressure, (iii) the liquids are reagents used for screening including emulsions, suspensions, and cell-free protein synthesis reagents, or (iv) each microwell comprises at least one filter hole, wherein the filter hole is smaller than a diameter of beads used with the system.

17-19. (canceled)

20. A method of identifying a nucleic acid molecule encoding a polypeptide and/or RNA having a desired bioactivity, the method comprising:
(a) attaching a plurality of nucleic acid constructs to a plurality of beads;
(b) loading the plurality of beads into microwells in the microwell array system of claim 1, wherein each microwell in the microwell array receives one or more beads;
(c) incubating the nucleic acid constructs with in vitro transcription/translation (IVTT) reagents for a time sufficient to produce a plurality of polypeptides encoded by the nucleic acid constructs in the microwell array;
(d) depositing nucleic acid constructs or polypeptides from each microwell in the microwell array at specific discrete locations on a substrate to form a blotting plate of nucleic acid constructs or polypeptides preserving the spatial relationship of the samples, wherein each location in the blotting plate has a known coordinate that corresponds to a specific microwell in the microwell array;
(e) determining a bioactivity of the polypeptides and/or RNA in the microwells or on the blotting plate and selecting a microwell or location on the blotting plate corresponding to a desired bioactivity; and
(g) determining which nucleic acid constructs correspond to the selected microwell or location on the blotting plate corresponding to the desired bioactivity, thereby identifying the nucleic acid construct that corresponds to the polypeptide and/or RNA having the desired bioactivity.

21. The method of claim 20, further comprising assembling the plurality of nucleic acid constructs in each microwell by releasing oligo fragments of the nucleic acid constructs and assembling the oligo fragments.

22. The method of claim 20, wherein:
(i) each bead is bound to one or more nucleic acid constructs,
(ii) the one or more nucleic acid constructs at the location on the substrate that corresponds to the microwell containing the polypeptide having the desired bioactivity is selected by light induced DNA trapping, light induced surface charge switch, light induced pH change, light induced dissociation, laser microdissection, micromanipulator, or other mechanic picking method,
(iii) the one or more nucleic acid constructs at the location on the substrate that corresponds to the microwell containing the polypeptide having the desired bioactivity is selected by sealing the nucleic acid construct by a sealing reagent,
(iv) the one or more nucleic acid constructs at the location on the substrate that corresponds to the microwell containing the polypeptide having the desired bioactivity is selected by hybridizing the nucleic acid construct with a set of fluorescence probes,
(v) the one or more nucleic acid constructs at the location of the substrate that corresponds to the microwell containing the polypeptide having the desired bioactivity is selected by a light-activated nuclease that releases the one or more nucleic acid constructs into solution for collection and sequencing to identify the constructs that correspond to the polypeptides that exhibit the desired bioactivity,
(vi) the one or more nucleic acid constructs at the location of the substrate that corresponds to the microwell containing the polypeptide having the desired bioactivity is selected automatically by the polypeptide catalyze a reaction that generates air bubble to expel liquid containing nucleic acid out from the microwells,
(vii) the one or more nucleic acid constructs at the location of the substrate that corresponds to the microwell containing the polypeptide having the desired bioactivity is selected automatically by the polypeptide catalyze a reaction or condition that deforms or dissolves the beads so that nucleic acid could passing through the filtering holes, or
(viii) the bioactivity of the polypeptide is analyzed by a catalytical reaction, a binding assay, and a cleavage assay resulting optical signals.

23-39. (canceled)

40. A method of selectively releasing one or more nucleic acid constructs from a substrate, the method comprising:
(a) providing a substrate comprising an array of nucleic acid constructs;
adding a photosensitive agent to the substrate;
exposing one or more selected locations on the substrate to light, wherein the light induces the photosensitive agent to cross-link to form a polymer layer at the selected locations, thereby trapping nucleic acid constructs at the selected locations within the substrate; and
washing the substrate with a wash solution, thereby releasing one or more nucleic acid constructs from unselected locations, or
(b) providing a surface comprising an array of nucleic acid constructs, wherein the nucleic acid constructs are attached to the surface through an electronic charge interaction; and
exposing one or more selected locations on the surface to light, wherein the light induces charge-switching of the surface, thereby releasing nucleic acid constructs at the selected locations on the surface.

41. The method of claim 40, wherein the one or more selected locations are exposed to light by using a light projector with a predetermined pattern.

42. The method of claim 40, wherein the substrate or surface is covered by a photomask, and the one or more selected locations are exposed to light by uncovering portions of the photomask at the selected locations.

43. The method of claim 40, further comprising sequencing the one or more nucleic acid constructs that are, for part (a) in the wash solution or, for part (b) released from the plate.

44. The method of claim 40, further comprising releasing and sequencing the nucleic acid constructs that are, for part (a), trapped by the cross-linked polymer or, for part (b), at unselected locations.

45-49. (canceled)

50. A method for loading of beads into microwells such that microwells contain either one or no beads, but that a low percentage of the microwells contain two or more beads, the method comprising
obtaining a plurality of beads in a liquid;
obtaining a microwell array system of claim 1, wherein each microwell comprises one or more larger filter holes and one or more smaller filter holes;
wherein each larger filter hole has a diameter that is smaller than a smallest outer diameter of the plurality of beads and is sized to enable the beads seat within and block the larger filter holes thereby decreasing flow of the liquid through the larger filter holes;
wherein each smaller filter hole has a diameter that is smaller than the diameter of the larger filter holes and sufficiently smaller than the smallest outer diameter of the plurality of beads such that the beads cannot block the flow of the liquid through the smaller filter holes; and
wherein blocking of the larger filter holes by one bead automatically prevents any additional bead from entering the microwell because of a decreased flow rate of the liquid through the microwell, while the smaller filter holes enable the liquid to drain slowly from the microwell to relieve pressure and to inhibit the beads from unblocking the one or more larger filter holes.

51. A method of selectively trapping or releasing targets in one or more microwells of interest on a microwell array, the method comprising:
identifying one or more microwells of interest; and
(i) in the case of trapping targets, selectively exposing the one or more microwells of interest to light to induce polymerization of a polymer solution in the one or more microwells of interest,
thereby trapping targets in the one or more microwells of interest, or
(ii) in the case of releasing targets, selectively exposing the microwells on the array to light except the one or more microwells of interest, wherein targets in microwells on the array except the one or more microwells of interest are trapped in the microwells due to polymerization of a polymer solution; and
collecting targets from the one or more microwells of interest.

52. The method of claim 51, wherein (i) identifying one or more microwells of interest comprises analyzing florescent signals from the microwell array, (ii) the one or more microwells of interest are exposed to light using a photomask, (iii) the one or more microwells of interest are exposed to light using a projector, or (iv) the targets are beads, nucleic acid constructs, or proteins.

53-60. (canceled)
